(19) United States
(12) Patent Application Publication
(10) Pub. No.: US 2023/0338609 A1
(43) Pub. Date: Oct. 26, 2023

(54) MEDICAL DEVICES AND MATERIALS COMPRISING BIODEGRADABLE POLYESTERS

(71) Applicant: AMICOAT AS, Tromsø (NO)

(72) Inventors: Wenche Stensen, Kvaløysletta (NO); John Sigurd Svendsen, Kvaløysletta (NO)

(73) Assignee: AMICOAT AS, Tromsø (NO)

(21) Appl. No.: 18/001,396

(22) PCT Filed: Jun. 14, 2021
(86) PCT No.: PCT/EP2021/065984

§ 371 Date: Dec. 9, 2022

(30) Foreign Application Priority Data
Jun. 12, 2020 (GB) 2008980.1

(51) Int. Cl. A61L 17/12; A61L 17/00; A61K 38/06; A61K 47/34; A61P 31/04

(52) U.S. Cl. CPC A61L 17/12; A61L 17/005; A61K 38/06; A61K 47/34; A61P 31/04; A61L 2300/406; A61L 2300/604; A61L 2420/02; A61L 2420/06; A61L 2300/606; A61L 2300/25

(57) ABSTRACT

The present invention provides a formulation comprising a biodegradable polyester compounded with a compound of Formula (I): AA-AA-AA-X-Y. The invention further provides methods of making these formulations, medical devices such as sutures comprising said formulations and methods of making said devices.

The present invention relates to antimicrobial formulations, in particular to formulations comprising polymers compounded with a small peptide or peptide-like molecule. These formulations find use in particular in medical devices.

The use of medical devices such as catheters, orthopaedic devices and other implants as well as surgical fasteners such as sutures etc. has increased. Despite improvements in device design and surgical procedures, infections associated with such medical devices are still a major concern. Conventional antibiotic treatment often fails due to the low level of antibiotic at the actual site of infection. The presence of biofilms on the introduced biomaterial/device can impair the efficacy of antibiotic treatment as can the existence of drug resistant strains.

Antibiotic releasing coatings are known for medical devices such as sutures and catheters. However, a patient may be infected with a resistant bacterium and due to the pattern of local release, a concentration gradient of the antibiotic exists which increases the risk of competitive selection for resistant bacteria. In particular, sutures impregnated with triclosan are marketed, but it is recognised that this antibiotic has been overused and has led to the development of resistant bacterial strains, moreover it is known to contribute to cross resistance to other antibiotics. Therefore there is pressure from health authorities to limit its use. While studies have shown that triclosan-coated sutures reduce the occurrence of surgical site infections, there is limited data for sutures coated with other active agents such as chlorhexidine (Onesti et al. 2018 European Review for Medical and Pharmacological Sciences 22: pages 5729-5739).

Wound infections after surgery are the third most common hospital acquired infections in the United States. Surgical site infections cause major discomfort for the patient, are potentially life threatening events, and prolong hospitalisation stays. Microbial adherence to the surface of sutures and other surgical fasteners has been recognised as one of the reasons for the development of incision infections.

Thus, there is a clear need for alternative sutures and other medical devices which can act to provide controlled release of an antimicrobial agent as well as limiting colonisation of the device itself.

The present inventors have found that they are able to combine certain small antimicrobial peptides with biodegradable polyesters to provide a material which can be used per se (e.g. a device made by 3D printing) or as a coating to another medical device.

Antimicrobial peptides are promising candidates as new antimicrobial agents as they are active against a broad spectrum of planktonic bacteria and biofilms, including antibiotic resistant strains. Moreover, bacteria are less likely to develop resistance to these rapidly acting peptides due to their mode of action, which includes disruption of the lipid membrane, rather than acting on a protein target. However, formulating peptides into substrates which can provide controlled release is not straightforward, peptides are generally poorly soluble compared to other classes of pharmaceutical and are usually degraded at the kinds of temperatures which may be required for blending or compounding the peptide into the substrate.

The present inventors have prepared material which can be used to provide controlled release of an active antimicrobial agent, i.e. the antimicrobial agent is leachable and can inhibit bacterial growth in the surrounding environment. The antimicrobial agent also acts to control microbial growth within the material and/or on the device which is coated with the blend of polymer and antimicrobial agent.

Thus, in one aspect, the present invention provides a formulation comprising a biodegradable polyester compounded with a compound of formula (I)

AA-AA-AA-X-Y (I)
wherein, in any order, 2 of said AA (amino acid) moieties are cationic amino acids, preferably lysine or arginine but may be histidine or any non-genetically coded or modified amino acid carrying a positive charge at pH 7.0, and 1 of said AA is an amino acid with a large lipophilic R group, the R group having 14-27 non-hydrogen atoms and preferably containing 2 or more, e.g. 2 or 3, cyclic groups which may be fused or connected, these cyclic groups will typically comprise 5 or 6 non-hydrogen atoms, preferably 6 non-hydrogen atoms (in the case of fused rings of course the non-hydrogen atoms may be shared);
X is a N atom, which may be but preferably is not substituted by a branched or unbranched C₁-C₁₀ alkyl or aryl group, e.g. methyl, ethyl or phenyl, and this group may incorporate up to 2 heteroatoms selected from N, O and S; and
Y is selected from the group consisting of R₁-R₂-R₃,
R₁-R₂-R₂-R₃, R₂-R₂-R₁-R₃, R₁-R₃ and R₄
wherein:
R₁ is C, O, S or N, preferably O;
R₂ is O;
each of R₁ and R₂ may be substituted by C₁-C₄ alkyl groups or unsubstituted, preferably Y is -R₁-R₂-R₃ (in which R₁ is preferably C) and preferably this group is not substituted, when Y is -R₁-R₂-R₂-R₃ or R₂-R₂-R₁-R₃ then preferably one or more of R₁ and R₂ is substituted;
R₃ is a group comprising 1 to 3 cyclic groups each of 5 or 6 non-hydrogen atoms (preferably all C atoms but optionally also containing N, O or S), 2 or more of the cyclic groups may be fused; one or more of the rings may be substituted and these substitutions may, but will typically not, include polar groups, suitable substituting groups include halogens, preferably bromine or fluorine and C₁-C₄ alkyl groups; R₃ incorporates a maximum of 15 non-hydrogen atoms, preferably 5-12, most preferably it is phenyl; and
R₄ is an aliphatic moiety having 2-20 non-hydrogen atoms, preferably these are carbon atoms but oxygen, nitrogen or sulphur atoms may be incorporated, preferably R₄ comprises 3-10, most preferably 3-6 non-hydrogen atoms and the moiety may be linear, branched or cyclic. If the R₄ group comprises a cyclic group this is preferably attached directly to the nitrogen atom of X.

Preferred compounds incorporate an R₄ group which is linear or branched, in particular a linear or branched alkyl group including ethyl, propyl, isopropyl, butyl, isobutyl, sec-butyl, tert-butyl, pentyl and isomers thereof, hexyl and isomers thereof etc.; propyl, isopropyl, butyl and isobutyl are especially preferred.

In some embodiments, R₄ is an aliphatic moiety (preferably an alkyl group) having 6-16 non-hydrogen atoms, preferably these are carbon atoms but oxygen, nitrogen or sulphur atoms may be incorporated, and the moiety may be linear, branched or cyclic.

In some preferred embodiments, R₄ is an isopropyl group.

Of the R₄ groups which comprise a cyclic group, preferred are molecules in which R₄ is cyclohexyl or cyclopentyl.

Suitable non-genetically coded amino acids and modified amino acids which can provide a cationic amino acid include analogues of lysine, arginine and histidine such as homolysine, ornithine, diaminobutyric acid, diaminopimelic acid, diaminopropionic acid and homoarginine as well as trimethylysine and trimethylornithine, 4-aminopiperidine-4-carboxylic acid, 4-amino-1-carbamimidoylpiperidine-4-carboxylic acid and 4-guanidinophenylalanine.

The large lipophilic R group of the AA may contain hetero atoms such as O, N or S, typically there is no more than one heteroatom, preferably it is nitrogen. This R group will preferably have no more than 2 polar groups, more preferably none or one, most preferably none.

The compounds, which are preferably peptides, are preferably of formula (II)

AA₁-AA₂-AA₁-X-Y (II)
wherein:
AA₁ is a cationic amino acid, preferably lysine or arginine but may be histidine or any non-genetically coded or modified amino acid carrying a positive charge at pH 7.0;
AA₂ is an amino acid with a large lipophilic R group, the R group having 14-27 non-hydrogen atoms and preferably containing 2 or more, e.g. 2 or 3, cyclic groups which may be fused or connected, these cyclic groups will typically comprise 5 or 6 non-hydrogen atoms, preferably 6 non-hydrogen atoms; and
X and Y are as defined above.

Further preferred compounds include compounds of formulae (III) and (IV):

AA₂-AA₁-AA₁-X-Y (III)

AA₁-AA₁-AA₂-X-Y (IV)
wherein AA₁, AA₂, X and Y are as defined above. Molecules of formula (II) are more preferred.

From amongst the above compounds certain are particularly preferred. In particular, compounds wherein the amino acid with a large lipophilic R group, conveniently referred to herein as AA₂, is tributyl tryptophan (Tbt) or a biphenylalanine derivative such as Phe (4-(2-Naphthyl)) [also referred to herein as Bip (4-(2-Naphthyl)], Phe (4-(1-Naphthyl)) [also referred to herein as Bip (4-(1-Naphthyl)], Bip (4-n-Bu), Bip (4-Ph) or Bip (4-T-Bu); Phe (4-(2-Naphthyl)) and Tbt being most preferred. In some preferred embodiments, the amino acid with a large lipophilic R group is tributyl tryptophan (Tbt).

Another preferred group of compounds are those wherein Y is -R₁-R₂-R₃ as defined above, preferably wherein R₁ and R₂ are unsubstituted, most preferably wherein R₁ and R₂ are both carbon atoms.

A further preferred group of compounds are those in which -X-Y together is the group —NHCH₂CH₂Ph.

The compounds include all enantiomeric forms, both D and L amino acids and enantiomers resulting from chiral centers within the amino acid R groups and the C-terminal capping group “-X-Y”. β and γ amino acids as well as a amino acids are included within the term ‘amino acids’, as are N-substituted glycines which may all be considered AA units. The molecules of the invention include beta peptides and depsipeptides.

Most preferred compounds are the following:

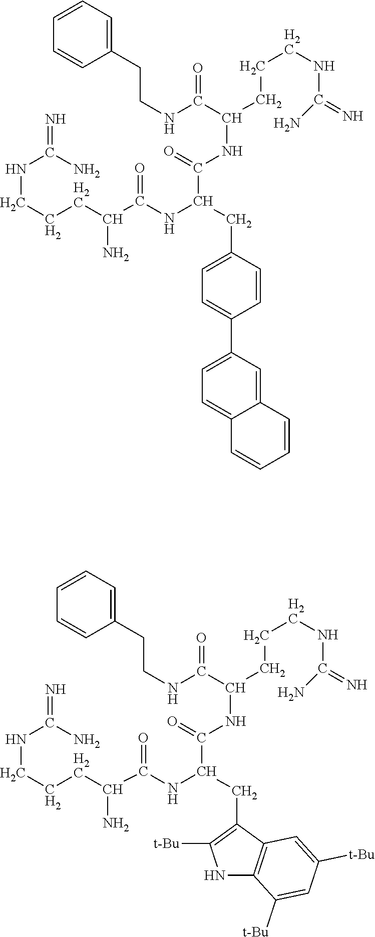 [SMILES: N=C(N)NCCCC(N)C(=O)NC(Cc1ccc(-c2ccc3ccccc3c2)cc1)C(=O)NC(CCCNC(=N)N)C(=O)NCCc1ccccc1 ; CC(C)(C)c1cc(C(C)(C)C)c2[nH]c(C(C)(C)C)c(CC(NC(=O)C(N)CCCNC(=N)N)C(=O)NC(CCCNC(=N)N)C(=O)NCCc3ccccc3)c2c1]

t-Bu represents a tertiary butyl group. This second compound incorporating the amino acid 2,5,7-Tris-tert-butyl-L-tryptophan is the most preferred compound of use in the present invention (and is also referred to herein as AMC-109). Analogues of this compound incorporating other cationic residues in place of Arg, in particular Lys, are also highly preferred. Analogues incorporating alternative C terminal capping groups as defined above are also highly preferred.

A further preferred group of compounds are those in which -X-Y together is selected from the group consisting of —NHCH(CH₃)₂, —NH(CH₂)₅CH₃, —NH(CH₂)₃CH₃, —NH(CH₂)₂CH₃, —NHCH₂CH(CH₃)₂, —NHcyclohexyl and -NHcyclopentyl, particularly preferred are compounds in which -X-Y is the group —NHCH(CH₃)₂ or —NH(CH₂)₅CH₃. A particularly preferred group of compounds are those in which -X-Y together is NHCH(CH₃)₂.

A preferred compound is a compound in which AA₁ is arginine, AA₂ is tributyl tryptophan and —X-Y together is NHCH(CH₃)₂.

Compounds of use in the present invention are preferably peptides.

The compounds of formulae (I) to (IV) may be peptidomimetics and peptidomimetics of the peptides described and defined herein also represent compounds of use in accordance with of the present invention. A peptidomimetic is typically characterised by retaining the polarity, three dimensional size and functionality (bioactivity) of its peptide equivalent but wherein the peptide bonds have been replaced, often by more stable linkages. By ‘stable’ is meant more resistant to enzymatic degradation by hydrolytic enzymes. Generally, the bond which replaces the amide bond (amide bond surrogate) conserves many of the properties of the amide bond, e.g. conformation, steric bulk, electrostatic character, possibility for hydrogen bonding etc. Chapter 14 of “Drug Design and Development”, Krogsgaard, Larsen, Liljefors and Madsen (Eds) 1996, Horwood Acad. Pub provides a general discussion of techniques for the design and synthesis of peptidomimetics. In the present case, where the molecule is reacting with a membrane rather than the specific active site of an enzyme, some of the problems described of exactly mimicking affinity and efficacy or substrate function are not relevant and a peptidomimetic can be readily prepared based on a given peptide structure or a motif of required functional groups. Suitable amide bond surrogates include the following groups: N-alkylation (Schmidt, R. et al., Int. J. Peptide Protein Res., 1995, 46, 47), retro-inverse amide (Chorev, M and Goodman, M., Acc. Chem. Res, 1993, 26, 266), thioamide (Sherman D. B. and Spatola, A. F. J. Am. Chem. Soc., 1990, 112, 433), thioester, phosphonate, ketomethylene (Hoffman, R. V. and Kim, H. O. J. Org. Chem., 1995, 60, 5107), hydroxymethylene, fluorovinyl (Allmendinger, T. et al., Tetrahydron Lett., 1990, 31, 7297), vinyl, methyleneamino (Sasaki, Y and Abe, J. Chem. Pharm. Bull. 1997 45, 13), methylenethio (Spatola, A. F., Methods Neurosci, 1993, 13, 19), alkane (Lavielle, S. et. al., Int. J. Peptide Protein Res., 1993, 42, 270) and sulfonamido (Luisi, G. et al. Tetrahedron Lett. 1993, 34, 2391).

The peptidomimetic compounds of the present invention will typically have 3 identifiable sub-units which are approximately equivalent in size and function to amino acids (AA units). The term ‘amino acid’ may thus conveniently be used herein to refer to the equivalent sub-unit of a peptidomimetic compound. Moreover, peptidomimetics may have groups equivalent to the R groups of amino acids and discussion herein of suitable R groups and of N and C terminal modifying groups applies, mutatis mutandis, to peptidomimetic compounds.

As is discussed in the text book referenced above, as well as replacement of amide bonds, peptidomimetics may involve the replacement of larger structural moieties with di- or tripeptidomimetic structures and in this case, mimetic moieties involving the peptide bond, such as azole-derived mimetics may be used as dipeptide replacements. Peptidomimetics and thus peptidomimetic backbones wherein the amide bonds have been replaced as discussed above are, however, preferred.

Suitable peptidomimetics include reduced peptides where the amide bond has been reduced to a methylene amine by treatment with a reducing agent e.g. borane or a hydride reagent such as lithium aluminium-hydride. Such a reduction has the added advantage of increasing the overall cationicity of the molecule.

Other peptidomimetics include peptoids formed, for example, by the stepwise synthesis of amide-functionalised polyglycines. Some peptidomimetic backbones will be readily available from their peptide precursors, such as peptides which have been permethylated, suitable methods are described by Ostresh, J. M. et al. in Proc. Natl. Acad. Sci. USA (1994) 91, 11138-11142. Strongly basic conditions will favour N-methylation over O-methylation and result in methylation of some or all of the nitrogen atoms in the peptide bonds and the N-terminal nitrogen.

Preferred peptidomimetic backbones include polyesters, polyamines and derivatives thereof as well as substituted alkanes and alkenes. The peptidomimetics will preferably have N and C termini which may be modified as discussed herein.

Compounds (e.g. peptides) of use in accordance with the present invention exhibit antimicrobial activity (typically antibacterial activity), in particular they exert a cytotoxic effect through a direct membrane-affecting mechanism and can be termed membrane acting antimicrobial agents. These compounds are lytic, destabilising or even perforating the cell membrane. This offers a distinct therapeutic advantage over agents which act on or intereact with proteinaceous components of the target cells, e.g. cell surface receptors. While mutations may result in new forms of the target proteins leading to antibiotic resistance, it is much less likely that radical changes to the lipid membranes could occur to prevent the cytotoxic effect. The lytic effect causes very rapid cell death and thus has the advantage of killing bacteria before they have a chance to multiply. In addition, the molecules may have other useful properties which kill or harm the target microbes e.g. an ability to inhibit protein synthesis, thus they may have multi-target activity.

The compounds for use in the invention may be synthesised in any convenient way. Generally the reactive groups present (for example amino, thiol and/or carboxyl) will be protected during overall synthesis. The final step in the synthesis will thus be the deprotection of a protected derivative of the invention.

In building up a peptide, one can in principle start either at the C-terminal or the N-terminal although the C-terminal starting procedure is preferred.

Methods of peptide synthesis are well known in the art but for the present invention it may be particularly convenient to carry out the synthesis on a solid phase support, such supports being well known in the art.

A wide choice of protecting groups for amino acids are known and suitable amine protecting groups may include carbobenzoxy (also designated Z) t-butoxycarbonyl (also designated Boc), 4-methoxy-2,3,6-trimethylbenzene sulphonyl (Mtr) and 9-fluorenylmethoxy-carbonyl (also designated Fmoc). It will be appreciated that when the peptide is built up from the C-terminal end, an amine-protecting group will be present on the α-amino group of each new residue added and will need to be removed selectively prior to the next coupling step.

Carboxyl protecting groups which may, for example be employed include readily cleaved ester groups such as benzyl (Bzl), p-nitrobenzyl (ONb), pentachlorophenyl (OPCIP), pentafluorophenyl (OPfp) or t-butyl (OtBu) groups as well as the coupling groups on solid supports, for example methyl groups linked to polystyrene.

Thiol protecting groups include p-methoxybenzyl (Mob), trityl (Trt) and acetamidomethyl (Acm).

A wide range of procedures exists for removing amine- and carboxyl-protecting groups. These must, however, be consistent with the synthetic strategy employed. The side chain protecting groups must be stable to the conditions used to remove the temporary α-amino protecting group prior to the next coupling step.

Amine protecting groups such as Boc and carboxyl protecting groups such as tBu may be removed simultaneously by acid treatment, for example with trifluoroacetic acid. Thiol protecting groups such as Trt may be removed selectively using an oxidation agent such as iodine.

Compounding (“compounded”) is a term used to describe the mixing and/or blending of two polymers or a polymer and one or more additives. This may conveniently be achieved by blending in a molten state or through the combining of multiple components in solution. Both of these approaches are described herein. The resultant mixture is ideally homogenous or nearly homogenous. For the avoidance of doubt, compounding does not involve a covalent attachment between the polyester and the compound of Formula (I), but rather compounding results in a non-covalent, releasable, association between the polyester and the compound of Formula (I). Thus, the compound may be considered to be releasably associated with the polyester.

Thus, the compound of Formula (I) is capable of being released from (or leaching from or diffusing out of) the formulation of the invention. This is important in the context of the present invention as compounds of Formula (I) have antimicrobial activity, and it is desirable that in use the compounds are capable of being released from the formulation to an area that requires an antimicrobial activity, for example to prevent or treat an infection of a wound, surgical site or site of an implantation of a medical device.

Preferably, in use, there is controlled (i.e. sustained) release of the compound of Formula (I) from the formulation. For example, there may be release of a therapeutically effective amount of the compound for at least 6, 8, 12 or 14 hours. A therapeutically effective amount will preferably result in delivery to the local environment of a concentration of the compound which is in excess of the Minimum Inhibitory Concentration (MIC) of the compound for the target bacteria. Release times may be extended by applying on top of the formulation of the invention (e.g. as an outer layer on a medical device) a layer of the biodegradable polyester which is not compounded with an active compound.

The ability of active compound to be released from the formulation of the invention may be readily determined by any suitable method, and the skilled person is familiar with such methods. A suitable method is described in the examples hereto. For example, sutures coated with a formulation in accordance with the present invention can be brought into contact with an agar plate that has been inoculated with bacteria (e.g. a bacteria of the genus Staphylococcus) and, after an appropriate incubation time, the plates can be inspected for the presence of a “zone of inhibition” (i.e. a zone with no bacterial growth or with reduced bacterial growth) around the suture. The presence of a “zone of inhibition” (e.g. as compared to a test with a control suture that does not contain an antimicrobial compound) is indicative that compound can be released from the formulation.

The formulation of the invention can be considered a controlled release formulation which releases the compound of Formula (I) in vivo. A compound of Formula (I) may be considered to be dispersed (releasably dispersed) through (or dispersed at least partially through) the polyester.

The compound of Formula (I) renders the polyester resistant to microbial colonization and biofilm formation. Therefore the coating and/or the device effectively keeps itself clean, which is highly advantageous as in-dwelling or other medical devices are often sites of microbial attachment and growth.

A liquid media test may be used to assess both the release of active compound and its anti-colonizing effect. A device coated with a polyester of the invention may be immersed into a liquid bacterial broth. The leakage efficacy (release) is determined by counting the number of surviving bacteria in the broth and the anti-colonizing efficacy is determined by counting the number of live bacteria adhered to the device after exposure to the bacterial broth.

The biodegradable polymers of use in the present invention are classed as polyesters as they contain repeating ester groups but may also comprise other functional groups. Typically they are terminated by ester groups but a free carboxylic acid group or an alkyl ester group may be used as end groups. Polymers capped with ester terminated and alkyl ester groups typically show longer degradation times than free carboxylic acid groups.

Particularly suitable polymers include polylactides (D or L forms), polyglycolides, polydioxanones and polycaprolactones. Co-polymers (including block co-polymers) incorporating these same monomers may also be used, e.g. co-polymers of D- and L-lactide (although L-lactide polymers are preferred), of lactide and glycolide and of lactide and caprolactone. A 50:50 enantiomeric mixture of poly (D,L-lactide-co-glycolide) is preferred. Polymers including co-polymers comprising trimethylene carbonate monomers may also be suitable. Monomer ratios in co-polymers may vary but co-polymers including lactide will typically contain 50% or more, e.g. at least 60% or %70 lactide monomers. The formulation may thus comprise a single polymer type, a single co-polymer or a mixture of one or more polymers and/or co-polymers.

The biodegradable polymers are typically synthetic. The use of biodegradable polymers is well known in the biomedical field. Biodegradable polymers of use in the present invention should be non-toxic and not be recognized as foreign by the patient. The products of biodegradation should also be non-cytotoxic and readily eliminated from the body.

Biodegradability is judged by the rate of degradation in use, i.e. in (or in contact with) the animal body. The biodegradable polyesters can also be termed bioresorbable (i.e. broken down and absorbed by the body). Degradation varies significantly from polymer to polymer but will typically be from 1 or 2 weeks to 4 years or more. Preferably, in situ, the polyester will have completely degraded in less than 6 months, e.g. within 2-4 months. Although in other preferred embodiments the polymer may persist for up to 4 years or even longer. The skilled person is aware of the degradation time of different polymers of interest and can select the polymer according to the intended use. For example, a suture may be desired to last for only a matter of a week or a few weeks, while some implants, e.g. orthopaedic or heart implants may remain in the body for years. Compounding with a compound of Formula (I) does not significantly affect the rate of degradation of the polymer.

Intrinsic viscosity (measured in dig) of the polymers will also vary between polymers, typically from 0.1 or 0.2 to 6 or 8, preferably from 0.5 to 2.5, more preferably 0.8 to 2.2, most preferably 0.8 to 1.2.

Molecular weight (weight average) will also depend on the polymer chosen and the particular medical application of interest. Typical molecular weights will be 3,000 to 30,000, e.g. 5,000 to 25,000. Although molecular weights up to 50,000, 80,000, 100,000 or more may be used in some scenarios. Preferred molecular weights are from 3,000 to 20,000, e.g. from 3,000 to 10,000 or 15,000.

Gel permeation chromatography may be used to measure both intrinsic viscosity and molecular weight.

Suppliers of suitable medical grade biodegradable polyesters include Evonik (their Resomer® range). Suitable polymers are readily available commercially and may be conveniently synthesized by a number of reactions including direct condensation of alcohols and acids, ring opening polymerizations and metal-catalyzed polymerization reactions.

Two distinct approaches may conveniently be employed in the production of formulations of the present invention.

Firstly, the polyester and the compound of Formula (I) may be blended by melting the two together. The polymer may be melted first and then the compound of Formula (I) added or the compound may be added prior to melting. The addition of the compound does not significantly impact on the melting point of the polymer. It is highly surprising that the compounds of formula (I), in particular the peptides of formula (I), can survive the heating which is necessary for compounding them with a biodegradable polyester. These molecules show unexpected thermostability.

Thus, in another aspect, the present invention provides a method of producing a formulation comprising a biodegradable polyester compounded with a compound of Formula (I), said method comprising melting the biodegradable polyester in admixture with a compound of Formula (I). Embodiments of other aspects of the invention described herein apply, mutatis mutandis, to this aspect of the invention.This compounding through melting is preferably performed using rapid heating, for example heating to 120-230 degrees centigrade (dependent on the m.p. of the polyester) in under 5 minutes, e.g. under 4 minutes, such as around 3 minutes. In this way the polymer melts before the peptide decomposes. The compound of Formula (I) is preferably only exposed to high temperatures (e.g. over 100 degrees) for up to 2 or 3 minutes (allowing for heat transfer into the mixture), The mixture is removed from the heat source as soon as the polyester has melted and mixed with the compound of Formula (I). In this embodiment, polyesters with melting points below around 230 degrees centigrade are preferred.

Alternatively, and preferably, the polymer and compound of Formula (I) may be compounded using solvents. In one embodiment the biodegradable polymer is dissolved in a first solvent and the compound of Formula (I) is dissolved in a second solvent which is miscible with the first solvent and then the two solutions are mixed. Both solvents are preferably organic solvents, preferably with some polar quality.

The two solvents must be selected to be miscible and to be able to dissolve the polymer and the compound of Formula (I) respectively. The solvents must also retain their ability to solvate their component when mixed, even miscible solvents when mixed may result in precipitation of one of the components. Suitable solvents for the polymer include chloroform, ethyl acetate, acetone and tetrahydrofuran (THF); polar aprotic solvents may be preferred. Suitable solvents for the compound of Formula (I) are more varied and include water, methanol, ethanol, chloroform, THF and DMSO. A preferred solvent for the polymer is ethyl acetate and for the compound of Formula (I) it is an alcohol, e.g. ethanol.

Alternatively, both the compound of Formula (I) and the polymer may be dissolved in a single solvent, either prior to mixing, at the time or mixing or after mixing. Suitable solvents for this approach include THF and chloroform. It may be necessary to combine the compound of Formula (I) with the solvent, e.g. THF, for a long time, e.g. at least 4 hours, preferably at least 12 hours, maybe 1 or more days, possibly up to 7 or 14 days. It is very surprising that a compound of Formula (I) such as AMC-109 is soluble in THF and chloroform.

Thus, in another aspect, the present invention provides a method of producing a formulation comprising a biodegradable polyester compounded with a compound of Formula (I), said method comprising forming a mixture of a compound of Formula (I), a biodegradable polyester and one or more solvents capable of dissolving said compound and said polyester and, optionally, drying said mixture.

In a preferred embodiment the present invention provides a method of producing a formulation comprising a biodegradable polyester compounded with a compound of Formula (I), said method comprising (i) providing a first solution comprising a compound of Formula (I); (ii) providing a second solution comprising a biodegradable polyester, wherein said second solution is miscible with the first solution; and (iii) mixing said first and second solutions. Embodiments of other aspects of the invention described herein apply, mutatis mutandis, to this aspect of the invention. The solvents used to produce the first and second solutions may be the same or different. Preferred solvents are described above.

After mixing the resultant mixture may be dried, this may be an active or passive process, a passive drying process may take several days, e.g. 1-6 days.

Another method of compounding the polyester with a compound of Formula (I) is electrospinning. Electrospinning is a well-known voltage-driven process governed by the electrohydrodynamic phenomena where fibres can be made from a polymer solution. To obtain a formulation in accordance with the present invention via an electrospinning method, a solution (or “spinning solution”) comprising the polyester and a compound of Formula (I) are electrospun into fibres comprising the compound. This solution may conveniently comprise two solvents as discussed above, which are miscible and individually able to dissolve either the polymer or the compound of Formula (I). Alternatively a single solvent such as THF may be used. Suitable electrospinning methods are as described in Scaffaro et al., European Polymer Journal 96 (2017), 266-277.

The amount of compound of Formula (I) in the compounded polymer formulation, on a w/w basis, may be as little as 2 or 3% up to 25 or 30%, typically 4 to 15 or 20%.

The formulation of the invention is preferably added as a coating to a medical device, although some medical devices may consist of formulations of the invention or medical devices may have sections or components made of the formulation, e.g. through 3D printing. Thus a further aspect of the present invention is a medical device coated, which includes partially coated, with a formulation of the invention as defined herein. Preferably the entire outer surface of the device is coated with a formulation of the invention. This coating results in a layer of the formulation on the surface (on any surface in contact with the body including with body fluids) of the device. The thickness of the layer will be chosen to provide desired functionality, in particular to provide a desired controlled release profile in situ.

In a yet further aspect of the invention is provided a formulation of the invention as defined herein which has been applied to a medical device.

Devices include sutures, surgical fasteners, catheters, lines etc. and implants including orthopedic implants such as hip and knee implants, as well as dental implants, pins, stents, cardiac rhythm devices and deep brain stimulation devices. Sutures are especially preferred.

The device to be coated may be dipped (perhaps several times, e.g. 3-10 times), in either a molten formulation of the invention or in a mixture comprising both the biodegradable polyester, a compound of Formula (I) and one or more solvents, and dried or allowed to dry. Suitable solvents and miscible solvent mixtures are as discussed herein. Dipping methods are particularly suited to devices such as sutures. Alternatively, a formulation of the invention may be applied to the medical device, e.g. an implant, by painting onto the surface of the device, e.g. by spray painting. Medical devices incorporating a formulation of the invention may also be produced by 3-D printing.

Suitable sutures to which the formulation of the invention may be applied include absorbable, optionally braided sutures. Such sutures may be made of nylon and include Ethicon, Surgilon and Nurolan sutures. It may be preferable to use a suture which is free of or has reduced levels of any coating other than the formulation of the invention. For example, sutures may be first treated to remove silicon coatings.

Absorbable (biodegradable) sutures are a preferred medical device according to the present invention and may be formed as follows. They are conveniently made of polyesters from monomers selected from the group comprising: lactic acid (both enantiomeric forms either alone or in combination), glycolic acid, caprolactone and dioxanone. To tailor the properties of the suture, it is common to copolymerise two (or more) of these monomers, or possibly bloc-polymerise shorter strands of different homopolymers. The most common polymers in absorbable sutures are copolymers of L-lactic acid and glycolic acid, PLGA, preferably made from around 90% glycolic acid and around 10% L-lactic acid. Polyglactin 910 is one such copolymer and has a high tensile strength and is conveniently used as the filament in absorbable sutures. The coating is preferably a 65/35 mole ratio of a lactide-glycolide copolymer (e.g. Polyglactin 370) applied in an amount of 2-10% of the filament mass. For handling properties an equal amount of calcium stearate may be added (i.e. equal with the coating polymer). The compound of Formula (I) is preferably compounded in the coating, or a further coating applied thereto, but may also be compounded in the filament.

Sutures of the invention may comprise 0.5-10 mg of a compound of Formula (I) per meter of suture length, preferably 1-5, e.g. 1-3 mg/m.

In another aspect, the invention provides a method of producing a medical device of the invention, said method comprising (i) providing a formulation comprising a biodegradable polyester compounded with a compound of Formula (I); and (ii) applying said formulation to the medical device (e.g. by dipping the device into said formulation or painting, e.g. spray painting, said formulation onto the device, or the core of the device such as the filament part of a suture). After application of the formulation to the medical device it may be actively dried (e.g. by modest heating or application of air currents) or allowed to dry. Embodiments of other aspects of the invention described herein apply, mutatis mutandis, to this aspect of the invention. If a solvent containing formulation is applied, solvent will evaporate during drying and leave a coating (or layer) comprising or consisting of a formulation of the invention on said medical device.

In an analogous method, the invention provides a method of producing a medical device of the invention, said method comprising (i) providing a formulation comprising a biodegradable polyester compounded with a compound of Formula (I); and (ii) applying said formulation to a backing sheet or other carrier. In this way a medical device consisting of, or consisting essentially of, the formulation of the invention may be prepared, for example to form a film, membrane, sheet or adhesive.

A further aspect provides a formulation comprising a biodegradable polyester compounded with a compound of Formula (I), or a medical device with such a formulation applied thereto, produced by a method of the invention. Embodiments of other aspects of the invention described herein apply, mutatis mutandis, to this aspect of the invention.

A further aspect of the present invention provides the formulation and devices of the invention for use in therapy.

“Therapy” includes treatment and prophylaxis, i.e. it includes both treatment and preventative uses.

In some embodiments, the invention provides a formulation or medical device of the invention for use in the treatment or prevention of an infection of a subject. In some preferred embodiments, the infection or potential infection is a surgical site infection or a wound infection, e.g. a wound or other site requiring closure by stitches or other surgical fasteners. In other preferred embodiments, the infection or potential infection is one associated with an implant (as discussed above) and includes biofilm formation on or around the device.

Preferably, the infection is a bacterial infection, for example an infection by Gram-positive bacteria (e.g. bacteria of the genus Staphylococcus or Streptococcus). In some embodiments, the infection is a Staphylococcus aureus infection. In some embodiments, the infection is a Staphylococcus epidermidis infection.

The formulations, devices, uses and methods of the invention are preferably effective (full or partial inhibition of bacterial growth in the surrounding environment through release of the active compound and/or a full or partial anticolonizing effect) against a broad spectrum of bacteria, particularly against Gram-positive and Gram-negative bacteria, e.g. they are effective against all of Staphylococcus aureus, Pseudomonas aeruginosa, Escherichia coli and Enterococcus faecium.

A further aspect of the present invention provides a formulation or medical device of the present invention for use in inhibiting bacterial growth in a subject. Embodiments of other aspects of the invention described herein apply, mutatis mutandis, to this aspect of the invention.

A further aspect of the invention provides a formulation or medical device of the present invention for use in therapy, preferably for use in the treatment or prevention of an infection of a subject. Embodiments of other aspects of the invention described herein apply, mutatis mutandis, to this aspect of the invention.

Alternatively viewed, the present invention provides a method of treating or preventing an infection which method comprises applying (or administering) to a subject in need thereof a formulation or medical device of the present invention. Embodiments of other aspects of the invention described herein apply, mutatis mutandis, to this aspect of the invention.

Alternatively viewed, the present invention provides a method of treating or preventing an infection which method comprises applying (or administering) to a subject in need thereof a therapeutically effective amount of a formulation or medical device of the present invention. Embodiments of other aspects of the invention described herein apply, mutatis mutandis, to this aspect of the invention.

A therapeutically effective amount will be determined based on the clinical assessment and the MIC values against target bacteria for the chosen compound of Formula (I).

A further aspect of the invention provides a compound of Formula (I) for use in therapy, preferably for use in the treatment or prevention of an infection of a subject, wherein said compound is administered to (or applied to) a subject as a formulation comprising a biodegradable polyester compounded with said compound, or in the form of a medical device with such a formulation applied thereto. Embodiments of other aspects of the invention described herein apply, mutatis mutandis, to this aspect of the invention.

The term “subject” or “patient” as used herein includes any mammal, for example humans and any livestock, domestic or laboratory animal. Specific examples include mice, rats, pigs, cats, dogs, sheep, rabbits, cows and monkey. Preferably, however, the subject or patient is a human subject. Thus, subjects or patients treated in accordance with the present invention will preferably be humans.

In some embodiments, subjects or patients are those having an infection, or those suspected of having an infection, or those at risk of having (or contracting) an infection. At risk patients are a preferred patient group and include those who require a medical implant or who must undergo surgery or need to have a surgical or other wound closed. For these patients, a medical device in accordance with the invention can be selected, e.g. a suture, other fastener or orthopaedic implant which incorporates a formulation of the invention. Such devices release the antimicrobial compound of Formula (I) to encourage an infection-free local environment in the patient's body around the device, and the presence of the compound of Formula (I) on/within the device inhibits colonization of the device itself by bacteria.

The invention also provides kits comprising one or more of the medical devices of the invention. Preferably said kits are for use in the therapeutic methods and uses described herein. Preferably said kits comprise instructions for use of the kit components. Preferably said kits are for treating or preventing infection, e.g. as described elsewhere herein, and optionally comprise instructions for use of the kit components to treat such infections.

As used throughout the entire application, the terms “a” and “an” are used in the sense that they mean “at least one”, “at least a first”, “one or more” or “a plurality” of the referenced components or steps, except in instances wherein an upper limit is thereafter specifically stated.

In addition, where the terms “comprise”, “comprises”, “has” or “having”, or other equivalent terms are used herein, then in some more specific embodiments these terms include the term “consists of” or “consists essentially of”, or other equivalent terms.

The invention will now be further described with reference to the following non-limiting Examples and Figures, in which:

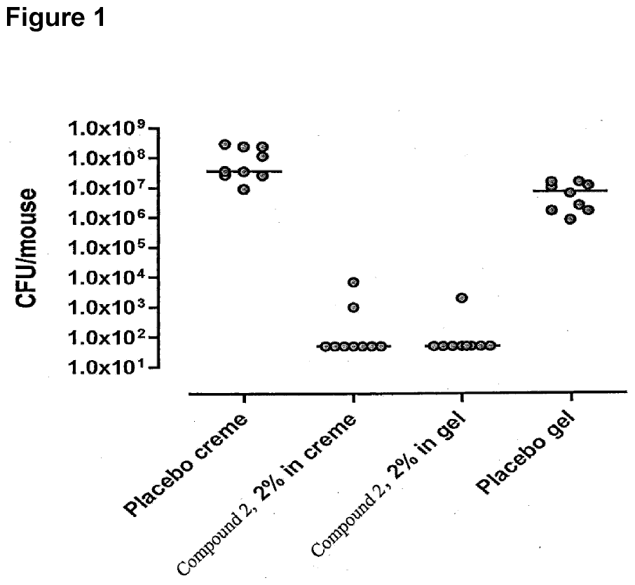

FIG. 1 is a graph showing the effect of one day topical treatment using compound 2 against Staphylococcus aureus FDA486 in a murine skin infection model. The number of colony forming units (CFU) are shown on the Y-axis and the type of topical treatment applied to the mice is shown on the X-axis. Compound 2 is also referred to herein as AMC-109.

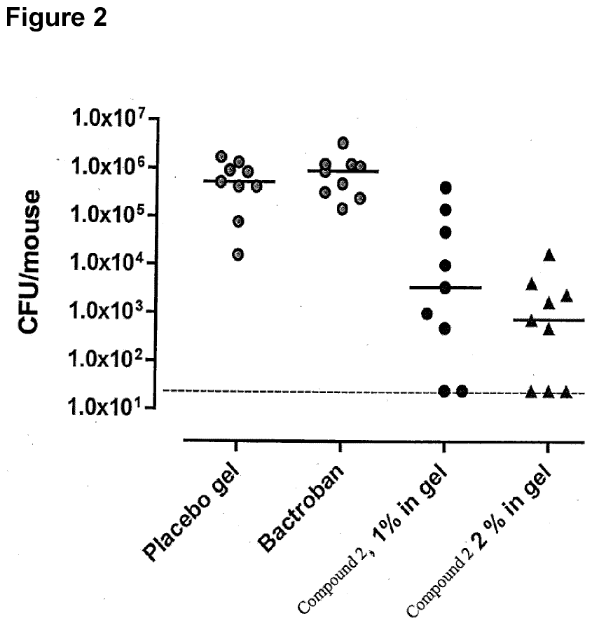

FIG. 2 is a graph showing the effect of one day topical treatment using compound 2 against Streptococcus pyogenes in a murine skin infection model. The number of colony forming units (CFU) are shown on the Y-axis and the type of topical treatment applied to the mice is shown on the X-axis. Compound 2 is also referred to herein as AMC-109.

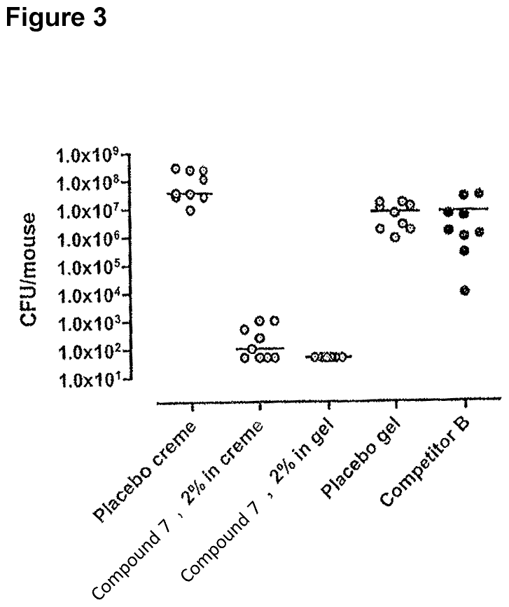

FIG. 3 is a graph showing the effect of one day topical treatment against S. aureus FDA486 in a murine skin infection model. Each mouse was treated at 9 am, 12 noon and 3 pm. The skin biopsy was collected at 6 pm. The median value is shown.

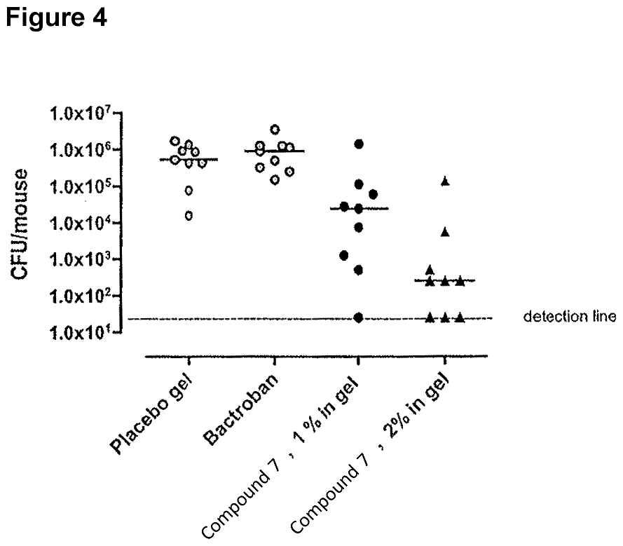

FIG. 4 is a graph showing the effect of one day topical treatment against Streptococcus pyogenes CS301 in a murine skin infection model. Each mouse was treated at 7 am, 10 am and 1 pm. The skin biopsy was collected at 4 pm. The median value is shown.

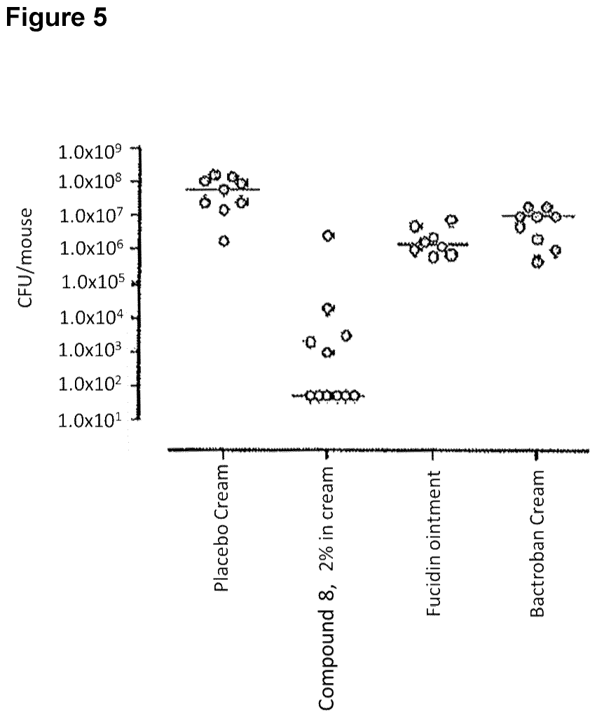

FIG. 5 is a graph showing the effect of one day topical treatment against S. aureus FDA486 in a murine skin infection model. Each mouse was treated at 9 am, 12 noon and 3 pm. The skin biopsy was collected at 6 pm. The median value is shown.

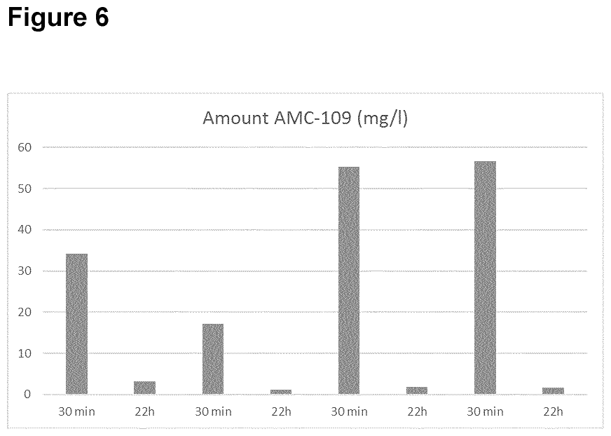

FIG. 6 is a graph showing quantitative analysis of the amount of AMC-109 released by extraction of the coated sutures. The first dataset is from the Test-01 batch, the next three datasets are three different sutures in the Test-02 batch.

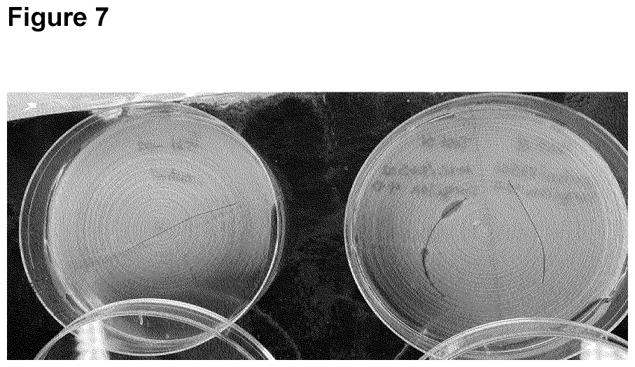

FIG. 7 is a photograph showing results from the growth inhibition assay on S. aureus, first time use.

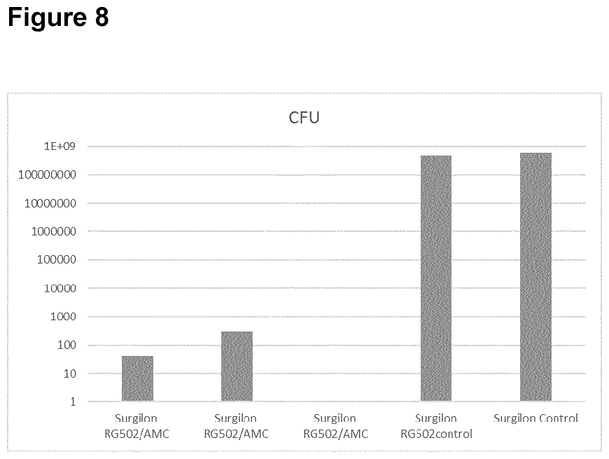

FIG. 8 is a graph showing the effect of AMC coated sutures on bacterial growth in liquid media measured by CFU in the growth media after exposure to the sutures.

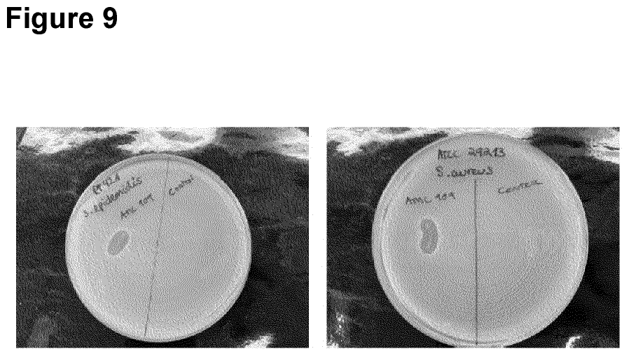

FIG. 9 is a photograph showing zones of inhibition as described in Example 9.

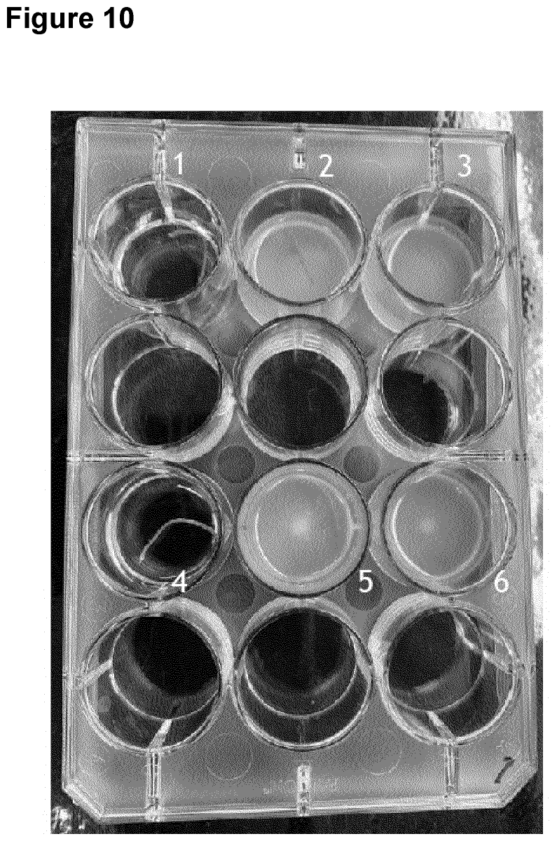

FIG. 10 is a photograph of the wells containing inoculated TSB in which the various sutures were placed. The numbered wells are as follows:
1: S. aureus Ethibond coated with AMC-109
2: S. aureus Ethibond non-coated
3: S. aureus growth control
4: S. epidermidis Ethibond coated with AMC-109
5: S. epidermidis Ethibond non-coated
6: S. epidermidis growth control

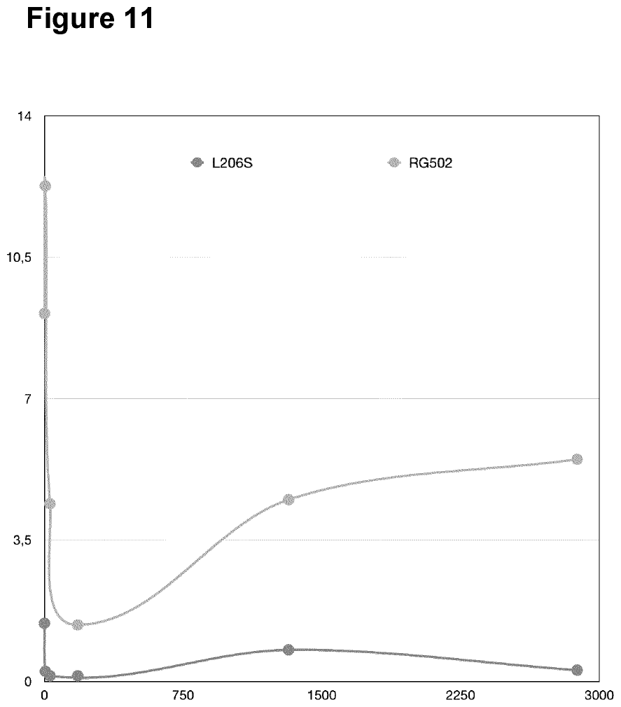

FIG. 11 is a graph showing leakage (in percentage of total amount) vs time (minutes) for AMC-109 containing bioresorbable polyesters RG502 and L206S.

### EXAMPLE 1

### Peptide Synthesis

### Chemicals

Protected amino acids Boc-Trp-OH, Boc-Arg-OH, Boc-4-phenyl-Phe and Ac-Arg-OH were purchased from Bachem AG while Boc-4-iodophenylalanine, Boc-3,3-diphenylalanine and Boc-(9-anthryl)alanine were purchased from Aldrich. Benzylamine, 2-phenylethylamine, 3-phenylpropylamine, (R)-2-phenylpropylamine, (S)-2-phenylpropylamine, N,N-methylbenzylamine, N,N-ethylbenzylamine and N,N-dibenzylamine making up the C-terminal of the peptide were purchased from Fluka except N-ethylbenzylamine which was purchased from Acros. Diisopropylethylamine (DI PEA), 1-hydroxybenzotriazole (1-HOBt), chlorotripyrrolidinophosphonium hexafluorophosphate (PyCloP) and O-(benzotriazol-1-yl)-N,N,N′,N′ tetramethyluronium hexafluorophosphate (HBTU) were purchased from Fluka. 4-n-Butylphenylboronic acid, 4-t-butylphenylboronic acid, 4-biphenylboronic acid, 2-napthylboronic acid, tri ortho-tolylphosphine, benzylbromide and palladium acetate were purchased from Aldrich. Solvents were purchased from Merck, Riedel-de Haën or Aldrich.

Preparation of Amino Acids

Preparation of Boc-2,5,7-tri-tert-butyltryptophan-OH: A mixture of H2N-Trp-OH (1.8 g, 8.8 mmol), t-BuOH (4.7 g, 63.4 mmol) in trifluoroacetic acid (19 mL) is stirred at 70 OC for 3 hours. The volume of the resulting mid-brown translucent solution is reduced on a rotary evaporator at room temperature for 30 min and then triturated by means of adding 60 mL of 7% (by weight) NaHCO3 drop-wise. The gray/white granular solid obtained is then recovered by vacuum filtration and dried in vacuo at room temperature for 24 hours. The product is isolated by crystallization from a near boiling mixture of 40% ethanol in water. Volumes typically are approximately 20 mL per gram of crude product.

A first crystallization from crude produces isolated product of 80-83% purity (HPLC) with respect to all other substances in the sample and approximately 94-95% purity with respect to the known TBT analogues. Yields at this stage are in the range 60-65%.

Benzylation of Boc-4-iodophenylalanine. Boc-4-iodophenylalanine (1 equivalent) was dissolved in 90% methanol in water and neutralized by addition of cesium carbonate until a weak alkaline pH (determined by litmus paper). The solvent was removed by rotary evaporation, and remaining water in the cesium salt of Boc-4-iodophenylalanine was further reduced by repeated azeotropic distillation with toluene. The resulting dry salt was dissolved in dimethylformamide (DMF), benzylbromide (1.2 equivalents) was added and the resulting mixture was stirred for 6-8 h. At the end of the reaction DMF was removed under reduced pressure and an oil containing the title compound is formed. This oil was dissolved in ethyl acetate and the resulting solution was washed with equal volumes of citric acid solution (three times), sodium bicarbonate solution and brine. The title compound was isolated as a pale yellow oil in 85% yield by flash chromatography using dichloromethane:ethyl acetate (95:5) as eluent. Crystalline benzyl Boc-4-iodophenylalanine could be obtained by recrystallisation from n-heptane.

General procedure for Suzuki couplings: Benzyl Boc-4-iodophenylalanine (1 equivalent), arylboronic acid (1.5 equivalents), sodium carbonate (2 equivalents), palladium acetate (0.05 equivalent) and tri ortho-tolylphosphine (0.1 equivalent) was added to a degassed mixture of dimethoxyethane (6 ml/mmol benzyl Boc-4-iodophenylalanine) and water (1 ml/mmol benzyl Boc-4-iodophenylalanine). The reaction mixture was kept under argon and heated to 80° C. for 4-6 h. After cooling to room temperature the mixture is filtered through a short pad of silicagel and sodium carbonate. The filter cake was further washed with ethyl acetate. The filtrates were combined and the solvents were removed under reduced pressure. The products were isolated by flash chromatography using mixtures of ethyl acetate and n-hexane as eluent.

Preparation of Boc-Bip(n-Bu)-OBn: The title compound was prepared in 53% yield from 4-n-butylphenylboronic acid using the general procedure for Suzuki couplings. Boc-Bip(n-Bu)-OBn was isolated using an 80:20 ethyl acetate:n-hexane eluent.

Preparation of Boc-Bip(t-Bu)-OBn: The title compound was prepared in 79% yield from 4-t-butylphenylboronic acid using the general procedure for Suzuki couplings. Boc-Bip(t-Bu)-OBn was isolated using an 80:20 ethyl acetate:n-hexane eluent.

Preparation of Boc-Bip(4-Ph)-OBn: The title compound was prepared in 61% yield from 4-biphenylboronic acid using the general procedure for Suzuki couplings. Boc-Bip(4-Ph)-OBn was isolated by recrystallisation of the crude product from n-heptane.

Preparation of Boc-Bip(4-(2-Naphtyl))-OBn: The title compound was prepared in 68% yield from 2-naphtylboronic acid using the general procedure for Suzuki couplings. Boc-Bip(4-(2-Naphtyl))-OBn was isolated by recrystallisation of the crude product from n-heptane.

Preparation of Boc-Bip(4-(1-Naphtyl))-OBn: The title compound was prepared from 2-naphtylboronic acid using the general procedure for Suzuki couplings. Boc-Bip(4-(1-Naphtyl))-OBn was isolated by recrystallisation of the crude product from n-heptane.

General procedure for deesterification of benzyl esters: The Benzyl ester is dissolved in DMF and hydrogenated for 2 days at ambient pressure using 10% Pd on carbon as catalyst. At the end of the reaction the catalyst is removed by filtration and the solvent is removed under reduced pressure. The free acids are isolated by recrystallisation from diethyl ether.

Preparation of Boc-Bip(4-n-Bu)-OH: The title compound was prepared in 61% yield from Boc-Bip(n-Bu)-OBn using the general procedure for deesterification.

Preparation of Boc-Bip(4-t-Bu)-OH: The title compound was prepared in 65% yield from Boc-Bip(t-Bu)-OBn using the general procedure for deesterification.

Preparation of Boc-Bip(4-Ph)-OH: The title compound was prepared in 61% yield from Boc-Bip(4-ph)-OBn using the general procedure for deesterification.

Preparation of Boc-Bip(4-(2-Naphtyl))-OH: The title compound was prepared in 68% yield from Boc-Bip(4-(2-Naphtyl))-OBn using the general procedure for deesterification.

Preparation of Boc-Bip(4-(2-Naphtyl))-OH: The title compound was prepared in 68% yield from Boc-Bip(4-(2-Naphtyl))-OBn using the general procedure for deesterification.

General procedure for Solution phase peptide synthesis using HBTU. The peptides were prepared in solution by stepwise amino acid coupling using Boc protecting strategy according to the following general procedure. The C-terminal peptide part with a free amino group (1 eq) and the Boc protected amino acid (1.05 eq) and 1-hydroxybenzotriazole (1-HOBt) (1.8 eq) were dissolved in DMF (2-4 ml/mmol amino component) before addition of diisopropylethylamine (DIPEA) (4.8 eq). The mixture was cooled on ice and O-(benzotriazol-1-yl)-N,N,N′,N′ tetramethyluronium hexafluorophosphate (HBTU) (1.2 eq) was added. The reaction mixture was shaken at ambient temperature for 1-2 h. The reaction mixture was diluted by ethyl acetate and washed with citric acid, sodium bicarbonate and brine. The solvent was removed under vacuum and the Boc protecting group of the resulting peptide was deprotected in the dark using 95% TFA or acetylchloride in anhydrous methanol.

Solution phase amide formation using PyCloP. Synthesis of Boc-Arg-N(CH₂Ph)₂. A solution of Boc-Arg-OH(1eq), NH(CH₂Ph)₂ (1.1 eq) and PyCloP (1 eq) in dry DCM (filtered through alumina)(2 ml) and DMF (1 ml). The solution was cooled on ice and DIPEA (2 eq) was added under stirring. The solution was stirred for 1 h at room temperature. The reaction mixture was evaporated, and redissolved in ethyl acetate and washed with citric acid, sodium bicarbonate and brine. The solvent was removed under vacuum and the Boc protecting group of the resulting peptide was deprotected in the dark using 95% TFA.

Peptide purification and analysis. The peptides were purified using reversed phase HPLC on a Delta-Pak (Waters) 018 column (100 Å, 15 μm, 25×100 mm) with a mixture of water and acetonitrile (both containing 0.1% TFA) as eluent. The peptides were analyzed by RP-HPLC using an analytical Delta-Pak (Waters) C₁₈ column (100 Å, 5 μm, 3.9×150 mm) and positive ion electrospray mass spectrometry on a VG Quattro quadrupole mass spectrometer (VG Instruments Inc., Altringham, UK).

### EXAMPLE 2

### In Vitro Activities of Peptides Defined Herein

### Materials and Methods

### Antimicrobials

Vials of pre-weighed Compound 1 and Compound 2 were supplied by Lytix Biopharma AS.

General compound formula: AA₁-AA₂-AA₁-XY

| | AA₁ | AA₂ | XY |
|---|---|---|---|
| Compound 1 | Arg | Phe(4-(2-Naphtyl)) | NHCH₂CH₂Ph |
| Compound 2 | Arg | 2,5,7-tri-tert- | NHCH₂CH₂Ph |
| | | butyltryptophan | |

### Bacterial Isolates

Bacterial isolates used in this study were from various sources worldwide stored at GR Micro Ltd. and maintained, with minimal sub-culture, deep frozen at −70° C. as a dense suspension in a high protein matrix of undiluted horse serum. The species used and their characteristics are listed in Table 1. These included 54 Gram-positive bacteria, 33 Gram-negative bacteria and 10 fungi.

### Determination of Minimum Inhibitory Concentration (MIC)

MICs were determined using the following microbroth dilution methods for antimicrobial susceptibility testing published by the Clinical and Laboratory Standards Institute (CLSI, formerly NCCLS):

M7-A6 Vol. 23 No. 2 January 2003 Methods for Dilution Antimicrobial Susceptibility Tests for Bacteria that Grow Aerobically; Approved Standard—Sixth Edition. M100-S15 Vol. 25 No 1. January 2005 Performance Standards for Antimicrobial Susceptibility Testing; Fifteenth Informational Supplement. M11-A6 Vol. 24 No. 2 Methods for Antimicrobial Susceptibility Testing of Anaerobic Bacteria; Approved Standard—Sixth Edition. M27-A2 Vol. 22 No. 15 Reference Method for Broth Dilution Antifungal Susceptibility Testing of Yeasts; Approved Standard—Second Edition. M38-A Vol. 22 No. 16 Reference Method for Broth Dilution Antifungal Susceptibility Testing of Filamentous Fungi; Approved Standard.

MIC estimations were performed using wet plates, containing the antibacterials or antifungals, prepared at GR Micro Ltd.

Cation-adjusted Mueller-Hinton broth (Oxoid Ltd., Basingstoke, UK and Trek Diagnostic Systems Ltd., East Grinstead, UK) (supplemented with 5% laked horse blood for Streptococcus spp., Corynebacterium jeikeium and Listeria monocytogenes) was used for aerobic bacteria, with an initial inoculum of approximately 10⁵ colony-forming units (CFU)/mL.

Haemophilus test medium (Mueller-Hinton broth containing 0.5% yeast extract and Haemophilus test medium supplement which contains 15 mg/L of each of haematin and NAD, all obtained from Oxoid Ltd., Basingstoke, UK) was used for the Haemophilus influenzae and inoculated with approximately 10⁵ CFU/mL.

Supplemented Brucella broth (SBB) was used for the anaerobic strains with an inoculum of approximately 10⁶ CFU/mL. SBB is a broth consisting of 1% peptone, 0.5% ‘Lab-lemco’, 1% glucose and 0.5% sodium chloride supplemented with 5 μg/L haemin and 1 μg/L vitamin K (both obtained from Sigma Aldrich Ltd.)

Yeast and filamentous fungal MIC were performed in MOPS buffered RPMI 1640 medium (MOPS buffer obtained from Sigma Aldrich Ltd., RPMI 1640 obtained from Invitrogen Ltd, Paisley, Scotland). The yeast inocula were in the range 7.5×10²-4×10³ CFU/mL and the filamentous fungi approximately 8×10³-1×10⁵ CFU/mL

Following normal practice all the plates containing Mueller-Hinton broth were prepared in advance, frozen at −70° C. on the day of preparation and defrosted on the day of use. Fungal, Haemophilus and anaerobic MIC determinations were all performed in plates prepared on the same day.

To evaluate whether freezing affected the activity of the peptides some MIC determinations were repeated using plates containing freshly-prepared Mueller-Hinton broth.

### Control Strains

The following control (reference) strains were included in the panel of strains tested

| | |
|---|---|
| Escherichia coli | ATCC 25922 |
| Staphylococcus aureus | ATCC 29213 |
| Enterococcus faecalis | ATCC 29212 |
| Streptococcus pneumoniae | ATCC 49619 |
| Pseudomonas aeruginosa | ATCC 27853 |
| Candida krusei | ATCC 6258 |

The control strains below were extra to the test strain panel and were included where appropriate, to check that the comparators were within range.

| | |
|---|---|
| Haemophilus influenzae | ATCC 49247 |
| Candida parapsilosis | ATCC 22019 |
| Bacteroides fragilis | ATCC 25285 |
| Eggerthella lenta | ATCC 43055 |

### Results

The results are shown in Table 1 as a single line listing. Repeat control strain results are shown in Table 2. It can be seen that the control strain results were highly reproducible including data from plates that contained Mueller Hinton broth either stored frozen or used fresh. Freezing plates also had no effect on the MIC for other bacterial strains.

The MIC data obtained is very encouraging and indicates that the peptides have quite a broad spectrum of activity.

TABLE 1
Single line list of the in vitro activity of two antimicrobial peptides and a comparator
against a panel of Gram-positive bacteria, Gram-negative bacteria and fungi.

| Species and properties | Compound 1 | Compound 2 |
|---|---|---|
| Candida albicans ATCC90028 - reference strain | 2 | 8 |
| Candida albicans ATCC24433 - reference strain | 2 | 8 |
| Candida tropicalis ATCC750 - reference strain | 2 | 4 |
| Candida parapsilosis ATCC90018 - reference strain | 4 | 16 |
| Candida (Issatchenkia) krusei ATCC6258 - reference strain | 4 | 4 |
| Aspergillus niger - G.R. Micro collection | 4 | 8 |
| Trichophyton mentagrophytes - G.R. Micro collection | 16 | 8 |
| Trichophyton interdigitale - G.R. Micro collection | 8 | 8 |
| Microsporum canis - G.R. Micro collection | 8 | 8 |
| Cryptococcus neoformans - G.R. Micro collection | 4 | 4 |
| Escherichia coli ATCC25922 - antibiotic-susceptible type strain | 32 | 8 |
| Escherichia coli ATCC32518 - β-lactamase positive type strain | 32 | 8 |
| Escherichia coli - multi-drug resistant clinical isolate | 32 | 8 |
| Klebsiella aerogenes NCTC11228 - antibiotic-susceptible type strain | 32 | 16 |
| Klebsiella aerogenes - multi-drug resistant clinical isolate | 64 | 16 |
| Enterobacter sp - antibiotic-susceptible clinical isolate | 32 | 4 |
| Enterobacter sp - multi-drug resistant clinical isolate | 64 | 16 |
| Pseudomonas aeruginosa ATCC27853 - antibiotic-susceptible type | 16 | 8 |
| [symbol] | | |
| Pseudomonas aeruginosa - multi-drug resistant clinical isolate | 32 | 4 |
| Stenotrophomonas maltophilia - antibiotic-susceptible clinical isolate | 64 | 8 |
| Salmonella sp - antibiotic-susceptible clinical isolate | 16 | 8 |
| Salmonella sp - multi-drug resistant clinical isolate | 16 | 8 |
| Shigella sp - antibiotic-susceptible clinical isolate | 32 | 8 |
| Morganella morganii - multi-drug resistant clinical isolate | ≥128 | 16 |
| Haemophilus influenzae - β- lactamase negative clinical isolate | ≥128 | 8 |
| Haemophilus influenzae - β -lactamase positive clinical isolate | ≥128 | 8 |
| Haemophilus influenzae β- lactamase negative ampicillin-resistant | ≥128 | 8 |
| [symbol] | | |
| Moraxella catarrhalis - β -lactamase positive clinical isolate | 4 | 4 |
| Moraxella catarrhalis - reduced fluoroquinolone susceptibility clinical | 8 | 8 |
| [symbol] | | |
| Acinetobacter baumanii - antibiotic-susceptible clinical isolate | 64 | 16 |
| Staphylococcus aureus ATCC 29213 - antibiotic-susceptible control | 4 | 2 |
| [symbol] | | |
| Staphylococcus aureus ATCC 25923 - antibiotic-susceptible control | 4 | 4 |
| [symbol] | | |
| Staphylococcus aureus ATCC 43300 - methicillin-resistant control strain | 4 | 2 |
| Staphylococcus aureus - methicillin-resistant clinical isolate | 4 | 4 |
| Staphylococcus aureus - multi-drug-resistant clinical isolate | 8 | 4 |
| Staphylococcus aureus - teicoplanin-intermediate clinical isolate | 4 | 4 |
| Staphylococcus epidermidis antibiotic susceptible clinical isolate | 16 | 8 |
| Staphylococcus epidermidis methicillin-resistant clinical isolate | 2 | 4 |
| Staphylococcus haemolyticus - antibiotic susceptible clinical isolate | 4 | 4 |
| Staphylococcus saprophyticus - antibiotic susceptible clinical isolate | 1 | 1 |
| Enterococcus faecalis - ATCC 29212 antibiotic-susceptible control | 4 | 4 |
| [symbol] | | |
| Enterococcus faecalis vancomycin-susceptible clinical isolate | 8 | 8 |
| Enterococcus faecalis vancomycin-resistant (VanA) clinical isolate | 16 | 8 |
| Enterococcus faecalis vancomycin-resistant (VanB) clinical isolate | 16 | 16 |
| Enterococcus faecalis high-level gentamicin-resistant clinical isolate | 16 | 8 |
| Enterococcus faecium vancomycin-susceptible clinical isolate | 8 | 8 |
| Enterococcus faecium vancomycin-resistant (VanA) clinical isolate | 16 | 8 |
| Enterococcus faecium vancomycin-resistant (VanB) clinical isolate | 8 | 4 |
| Enterococcus gallinarum vancomycin-resistant (VanC) clinical isolate | 4 | 4 |
| Streptococcus pneumoniae - ATCC 49619 antibiotic-susceptible control | 32 | 16 |
| [symbol] | | |
| Streptococcus pneumoniae - penicillin-susceptible clinical isolate | 64 | 32 |
| Streptococcus pneumoniae - penicillin-intermediate clinical isolate | 32 | 32 |
| Streptococcus pneumoniae - penicillin-resistant clinical isolate | 32 | 16 |
| Streptococcus pneumoniae - multi-drug resistant clinical isolate | 64 | 32 |
| Streptococcus pyogenes - Macrolide (MLS) resistant clinical isolate | 32 | 16 |
| Streptococcus pyogenes - Macrolide (M-type) resistance clinical isolate | 32 | 16 |
| Corynebacterium jeikeium - antibiotic-susceptible clinical isolate | 32 | 16 |
| Corynebacterium jeikeium - multi-drug resistant clinical isolate | 32 | 8 |
| Listeria monocytogenes - antibiotic-susceptible clinical isolate | 32 | 16 |
| MU50 Staphylococcus aureus (MRSA) - VISA type strain | 4 | 4 |
| EMRSA3 Staphylococcus aureus (MRSA) - SSCmec type 1 | 4 | 4 |
| EMRSA16 Staphylococcus aureus (MRSA) - SSCmec type 2 | 4 | 4 |
| EMRSA1 Staphylococcus aureus (MRSA) - SSCmec type 3 | 8 | 8 |
| EMRSA15 Staphylococcus aureus (MRSA) - SSCmec type 4 | 4 | 4 |
| HT2001254 Staphylococcus aureus (MRSA) - PVL positive | 4 | 4 |
| Streptococcus agalactiae - antibiotic-susceptible clinical isolate | 16 | 8 |
| Streptococcus agalactiae - macrolide-resistant clinical isolate | 32 | 16 |
| Group C Streptococcus - antibiotic-susceptible clinical isolate | 32 | 16 |
| Group C Streptococcus - macrolide-resistant clinical isolate | 64 | 32 |
| Group G Streptococcus - antibiotic-susceptible clinical isolate | 32 | 8 |
| Group G Streptococcus - macrolide-resistant clinical isolate | 32 | 16 |
| Streptococcus mitis - antibiotic-susceptible clinical isolate | 64 | 16 |
| Streptococcus mitis - macrolide-resistant clinical isolate | ≥128 | 32 |
| Streptococcus constellatus - antibiotic-susceptible clinical isolate | 64 | 32 |
| Streptococcus constellatus - macrolide-resistant clinical isolate | 64 | 32 |
| Streptococcus oralis - antibiotic-susceptible clinical isolate | 64 | 32 |
| Streptococcus oralis - macrolide-resistant clinical isolate | 32 | 32 |
| Streptococcus bovis - antibiotic-susceptible clinical isolate | 64 | 32 |
| Streptococcus bovis - macrolide-resistant clinical isolate | 8 | 8 |
| Streptococcus sanguis - antibiotic-susceptible clinical isolate | 64 | 32 |
| Streptococcus sanguis - macrolide-resistant clinical isolate | 32 | 32 |
| Clostridium perfringens - antibiotic-susceptible clinical isolate | ≥128 | 32 |
| Clostridium difficile - antibiotic-susceptible clinical isolate | 32 | 16 |

[symbol]

indicates data missing or illegible when filed

TABLE 2
In vitro activity of two antimicrobial peptides and comparators against ATCC control
strains (Including ATCC control strains extra to the test strain panel)

| Strain | | Compound | | |
|---|---|---|---|---|
| No. | Species and properties | 1 | 2 | Plate type |
| GP01 | Staphylococcus aureus ATCC 29213 | 8 | 4 | Frozen MHB |
| | antibiotic-susceptible control strain | | | |
| GP01 | Staphylococcus aureus ATCC 29213 | 4 | 4 | Frozen MHB |
| | antibiotic-susceptible control strain | | | |
| GP01 | Staphylococcus aureus ATCC 29213 | 4 | 2 | Fresh MHB |
| | antibiotic-susceptible control strain | | | |
| GN01 | Escherichia coli ATCC 25922 | 32 | 8 | Frozen MHB |
| | antibiotic-susceptible type strain | | | |
| GN01 | Escherichia coli ATCC 25922 | 32 | 8 | Frozen MHB |
| | antibiotic-susceptible type strain | | | |
| GN01 | Escherichia coli ATCC 25922 | 16 | 8 | Fresh MHB |
| | antibiotic-susceptible type strain | | | |
| GN10 | Pseudomonas aeruginosa ATCC 27853 | 16 | 8 | Frozen MHB |
| | antibiotic-susceptible type strain | | | |
| GN10 | Pseudomonas aeruginosa ATCC 27853 | 32 | 8 | Frozen MHB |
| | antibiotic-susceptible type strain | | | |
| GN10 | Pseudomonas aeruginosa ATCC 27853 | 8 | 8 | Fresh MHB |
| | antibiotic-susceptible type strain | | | |
| GP11 | Enterococcus faecalis - ATCC 29212 | 8 | 8 | Frozen MHB |
| | antibiotic-susceptible control strain | | | |
| GP11 | Enterococcus faecalis - ATCC 29212 | 8 | 8 | Frozen MHB |
| | antibiotic-susceptible control strain | | | |
| GP11 | Enterococcus faecalis - ATCC 29212 | 4 | 4 | Fresh MHB |
| | antibiotic-susceptible control strain | | | |
| | Haemophilus influenzae - ATCC 47247 | 32 | 4 | HTM |
| | Candida parapsilosis ATCC 22019 | 4 | 8 | RPMI 1640 |
| F05 | Candida (Issatchenkia) krusei ATCC 6258 | 8 | 8 | RPMI 1640 |
| | reference strain | | | |
| F05 | Candida (Issatchenkia) krusei ATCC 6258 | 8 | 8 | RPMI 1640 |
| | reference strain | | | |
| | Bacteroides fragilis - ATCC 25285 | 64 | 64 | SBB |
| | Eggerthella lenta - ATCC 43055 | 16 | 32 | SBB |

MHB, Mueller Hinton broth; HTM, haemophilus test medium; SBB, supplemented Brucella broth.

### EXAMPLE 3

### Stability Towards Tryptic Deciradation and Antimicrobial Activity

Compounds of formula AA₁-AA₂-AA₁-NHCH₂CH₂Ph were tested for their trypsin resistance and antimicrobial activity.

### Measurements and Calculation of Peptide Half-Life

Each peptide was dissolved in a 0.1 M NH₄HCO₃ buffer (pH 6.5) to yield a final peptide concentration of 1 mg/ml. A trypsin solution was prepared by dissolving 1 mg of trypsin in 50 ml 0.1 M NH₄HCO₃ buffer (pH 8.2). For the stability determination, 250 μl freshly made trypsin solution and 250 μl peptide solution were incubated in 2 ml of 0.1 M NH₄HCO₃ buffer (pH 8.6) at 37° C. on a rocking table. Aliquots of 0.5 ml were sampled at different time intervals, diluted with 0.5 ml water:acetonitrile (60:40 v/v) containing 1% TFA and analysed by RP-HPLC as described above. Samples without trypsin addition taken at 0 h and after 20 h at 37° C. were used as negative controls. Integration of the peak area at 254 nm for samples taken during the first 5 hours of the assay was used to generate the τ_{1/2}. Peptides that displayed no degradation during the first 24 h were classified as stable.

### Antibacterial Assay

MIC determinations on Staphylococcus aureus, strain ATCC 25923, Methicillin resistant Staphylococcus aureus (MRSA) strain ATCC 33591 and Methicillin resistant Staphylococcus epidermidis (MRSE) strain ATCC 27626 were performed by Toslab AS using standard methods. Amsterdam, D. (1996) Susceptibility testing of antimicrobials in liquid media, in Antibiotics in Laboratory Medicine. 4th ed (Lorian, V., Ed.) pp 75-78, Williams and Wilkins Co, Baltimore.

TABLE 3
Stability of AA₁-AA₂-AA₁-NHCH₂CH₂Ph peptides towards trypsin measured
as half-life (τ_{1/2}) and antibacterial activities displayed as MIC.

| | | | | MIC^{b} (μM) | | |
|---|---|---|---|---|---|---|
| Peptide | AA₁ | AA₂ | τ_{1/2}^{a} (h) | S. aureus^{c} | MRSA^{d} | MRSE^{e} |
| Compound 6^{f} | Arg | Trp | 7 | 145 | 97 | 81 |
| Compound 5 | Arg | Bip(4-Ph) | Stable | 5 | 3 | 3 |
| Compound 4 | Lys | 2,5,7-tri-tert- | Stable | | 3 | <2 |
| | | butyltryptophan | | | | |
| Compound 3 | Arg | Phe(4-(1- | 20 | | 3 | 3 |
| | | Naphtyl)) | | | | |
| Compound 2 | Arg | 2,5,7-tri-tert- | Stable | <3 | <3 | <3 |
| | | butyltryptophan | | | | |
| Compound 1 | Arg | Phe(4-(2- | Stable | 4 | <3 | <3 |
| | | Naphtyl)) | | | | |

^{a}Medical Calculator from Cornell University was used to calculate the half-life.
^{b}Minimal inhibitory concentration
^{c}Staphylococcus aures strain ATCC 25923
^{d}Methicillin resistant Staphylococcus aureus ATCC 33591
^{e}Methicillin resistant Staphylococcus epidermis ATCC 27626
^{f}not within compound definition for invention

### EXAMPLE 4

### In Vivo Activity of Compound 2

The skin of mice was infected with Staphylococcus aureus or Streptococcus pyogenes and subsequently given a total of three treatments at three hourly intervals. Three hours after the last treatment, skin biopsies were collected and the number of colony forming units (CFUs) present in the skin sample was determined. Results are shown in FIGS. 1 and 2, expressed as the number of colony forming units per mouse.

In experiment 1 (FIG. 1), compound 2 was applied to the murine skin as part of either a cream or a gel containing 2% (w/w) of compound 2. The same cream or gel without compound 2 was used as a negative control (placebo). It can clearly be seen that the number of CFUs was reduced when a cream or gel containing compound 2 was applied to the murine skin, compared to the negative control, indicating that compound 2 exerted an antimicrobial effect against Staphylococcus aureus. The nature of the carrier, cream or gel, had no significant effect.

In experiment 2 (FIG. 2), compound 2 was applied in two different concentrations, as either a 1% or a 2% gel. A placebo gel and a known antibacterial “bactroban” were used as controls. It can be seen that gels containing compound 2 were more effective at reducing the number of CFUs than the placebo gel or the bactroban. The gel containing 2% of compound 2 was more effective than the gel containing only 1% of compound 2.

### EXAMPLE 5

### Preparation and Physical, Antimicrobial and Aemolytic Properties of Compounds of Use in the Invention

Peptide Synthesis—Relevant Information is Also Provided in Example 1.

Chemicals:

Protected amino acids Boc-Arg-OH, and Boc-4-phenyl-Phe were purchased from Bachem AG while Boc-4-iodophenylalanine was purchased from Aldrich. isopropylamine, propylamine, hexylamine, butylamine, hexadecylamine, isobutylamine,cyclohexylamine and cyclopentylamine making up the C-terminal of the peptide were purchased from Fluka. Diisopropylethylamine (DIPEA), 1-hydroxybenzotriazole (1-HOBt), chlorotripyrrolidinophosphonium hexafluorophosphate (PyCloP) and O-(benzotriazol-1-yl)-N,N,N′,N′ tetramethyluronium hexafluorophosphate (HBTU) were purchased from Fluka. 4-n-Butylphenylboronic acid, 4-t-butylphenylboronic acid, 4-biphenylboronic acid, 2-napthylboronic acid, tri ortho-tolylphosphine, benzylbromide and palladium acetate were purchased from Aldrich. Solvents were purchased from Merck, Riedel-de Haën or Aldrich.

Preparation of Boc-Phe(4-4′-biphenyl)-OBn: The title compound was prepared in 61% yield from 4-biphenylboronic acid using the general procedure for Suzuki couplings. Boc-Phe(4-4′-biphenyl)-OBn was isolated by recrystallisation of the crude product from n-heptane.

Preparation of Boc-Phe(4-(2′-Naphtyl))-OBn: The title compound was prepared in 68% yield from 2-naphtylboronic acid using the general procedure for Suzuki couplings. Boc-Phe(4-(2′-Naphtyl))-OBn was isolated by recrystallisation of the crude product from n-heptane.

Preparation of Boc-Phe(4-4′-biphenyl)-OH: The title compound was prepared in 61% yield from Boc-Phe(4-4′-biphenyl)-OBn using the general procedure for deesterification.

Preparation of Boc-Phe(4-(2′-Naphtyl))-OH: The title compound was prepared in 68% yield from Boc-Phe(4-(2-Naphtyl))-OBn using the general procedure for deesterification.

General procedure for Solution phase peptide synthesis using HBTU is described in Example 1.

Solution phase amide formation using PyCloP is described in Example 1.

Peptide purification and analysis is described in Example 1.

TABLE 4
General compound formula: Arg-AA₂-Arg-X-Y

| | | | Purity |
|---|---|---|---|
| Compound | AA₂ | XY | (HPLC) |
| 7 | 2,5,7-tri-tert- | NHCH(CH₃)₂ | |
| | butyltryptophan | | |
| 8 | 2,5,7-tri-tert- | NH(CH₂)₅CH₃ | |
| | butyltryptophan | | |
| 9 | 2,5,7-tri-tert- | NH(CH₂)₃CH₃ | 87 |
| | butyltryptophan | | |
| 10 | 2,5,7-tri-tert- | NH(CH₂)₂CH₃ | 99 |
| | butyltryptophan | | |
| 11 | 2,5,7-tri-tert- | NH(CH₂)₁₅CH₃ | 80 |
| | butyltryptophan | | |
| 12 | 2,5,7-tri-tert- | NHCH₂CH(CH₃)₂ | 97 |
| | butyltryptophan | | |
| 13 | 2,5,7-tri-tert- | NHcyclohexyl | 95 |
| | butyltryptophan | | |
| 14 | 2,5,7-tri-tert- | NHcyclopentyl | 91 |
| | butyltryptophan | | |
| 15 | Phe(4-4′-biphenyl) | NHCH(CH₃)₂ | |
| 16 | Phe(4-4′-biphenyl) | NH(CH₂)₅CH₃ | |
| 17 | Phe(4-(2′-Naphtyl)) | NHCH(CH₃)₂ | |
| 18 | Phe(4-(2′-Naphtyl)) | NH(CH₂)₅CH₃ | |

### Antimicrobial Assay

MIC determinations on Staphylococcus aureus, strain ATCC 25923, Methicillin resistant Staphylococcus aureus (MRSA) strain ATCC 33591 and Methicillin resistant Staphylococcus epidermidis (MRSE) strain ATCC 27626 were performed by Toslab AS using standard methods. Amsterdam, D. (1996) Susceptibility testing of antimicrobials in liquid media, in Antibiotics in Laboratory Medicine. 4th ed (Lorian, V., Ed.) pp 75-78, Williams and Wilkins Co, Baltimore.

TABLE 5
Antimicrobial and toxic properties of compounds of use in the invention

| | C. albicans | S. aureus | MRSA | MRSE | S. pyogenes | E. coli | P. aeruginosa | |
|---|---|---|---|---|---|---|---|---|
| Compound | (mg/L) | (mg/L) | (mg/L) | (mg/L) | (mg/L) | (mg/L) | (mg/L) | EC50 |
| 7 | 25 | <2 | <2 | <2 | <2 | 7 | 7 | 720 |
| 8 | 5 | 2 | 2 | <1 | 2 | 5 | 5 | 32 |
| 9 | 10 | 2 | 3 | <2 | 2 | | | 350 |
| 10 | 10 | 2 | 3 | <2 | 2 | | | 620 |
| 11 | >100 | 5 | 4 | 4 | 6 | >100 | >100 | 38 |
| 12 | 10 | <2 | 3 | 2 | 2 | | | 300 |
| 13 | 10 | <2 | 2 | 2 | <2 | | | 55 |
| 14 | 10 | <2 | >15 | <2 | 2 | | | 340 |

### EXAMPLE 6

### In Vitro Broad Panel Screening of Selected Compounds

### Materials and Methods

### Antimicrobials

Vials of pre-weighed Compound 7 and Compound 8 were supplied by Lytix Biopharma AS.

General compound formula: AA₁-AA₂-AA₁-X-Y

| | AA₁ | AA₂ | XY |
|---|---|---|---|
| Compound 7 | Arg | 2,5,7-tri-tert- | NHCH(CH₃)₂ |
| | | butyltryptophan | |
| Compound 8 | Arg | 2,5,7-tri-tert- | NH(CH₂)₅CH₃ |
| | | butyltryptophan | |

### Bacterial Isolates

Bacterial isolates used in this study are as described in Example 2.

### Determination of Minimum Inhibitory Concentration (MIC)

MICs were determined as described in Example 2.

### Results

The results are shown in Table 6 as a single line listing.

The MIC data obtained is very encouraging and indicates that the peptides have quite a broad spectrum of activity.

TABLE 6
Single line list of the in vitro activity of two antimicrobial peptides against
a panel of Gram-positive bacteria, Gram-negative bacteria and fungi.

| | Compound 7 | Compound 8 |
|---|---|---|
| Species and properties | (mg/L) | (mg/L) |
| Candida albicans ATCC90028 - reference strain | 32 | 4 |
| Candida albicans ATCC24433 - reference strain | 64 | 8 |
| Candida tropicalis ATCC750 - reference strain | 4 | 4 |
| Candida parapsilosis ATCC90018 - reference strain | 64 | 8 |
| Candida (Issatchenkia) krusei ATCC6258 - reference strain | 8 | 32 |
| Aspergillus niger - G.R. Micro collection | 32 | 4 |
| Trichophyton mentagrophytes - G.R. Micro collection | 8 | 4 |
| Trichophyton interdigitale - G.R. Micro collection | 16 | 4 |
| Microsporum canis - G.R. Micro collection | 16 | 4 |
| Cryptococcus neoformans - G.R. Micro collection | 8 | 2 |
| Escherichia coli ATCC25922 - antibiotic-susceptible type strain | 32 | 4 |
| Escherichia coli ATCC32518 - β-lactamase positive type strain | 32 | 8 |
| Escherichia coli - multi-drug resistant clinical isolate | 32 | 8 |
| Klebsiella aerogenes NCTC11228 - antibiotic-susceptible type strain | 64 | 8 |
| Klebsiella aerogenes - multi-drug resistant clinical isolate | 32 | 8 |
| Enterobacter sp - antibiotic-susceptible clinical isolate | 64 | 8 |
| Enterobacter sp - multi-drug resistant clinical isolate | ≥128 | 8 |
| Pseudomonas aeruginosa ATCC27853 - antibiotic-susceptible type strain | 32 | 8 |
| Pseudomonas aeruginosa - multi-drug resistant clinical isolate | 8 | 4 |
| Stenotrophomonas maltophilia - antibiotic-susceptible clinical isolate | 32 | 4 |
| Salmonella sp - antibiotic-susceptible clinical isolate | 16 | 8 |
| Salmonella sp - multi-drug resistant clinical isolate | 16 | 8 |
| Shigella sp - antibiotic-susceptible clinical isolate | 32 | 4 |
| Morganella morganii - multi-drug resistant clinical isolate | 32 | 8 |
| Haemophilus influenzae - β- lactamase negative clinical isolate | 32 | 16 |
| Haemophilus influenzae - β -lactamase positive clinical isolate | 16 | 4 |
| Haemophilus influenzae β- lactamase negative ampicillin-resistant clinical isolate | 16 | 8 |
| Moraxella catarrhalis - β -lactamase positive clinical isolate | 4 | 16 |
| Moraxella catarrhalis - reduced fluoroquinolone susceptibility clinical isolate | 8 | 16 |
| Acinetobacter baumanii - antibiotic-susceptible clinical isolate | 64 | 16 |
| Staphylococcus aureus ATCC 29213 - antibiotic-susceptible control strain | 8 | 4 |
| Staphylococcus aureus ATCC 25923 - antibiotic-susceptible control strain | 8 | 4 |
| Staphylococcus aureus ATCC 43300 - methicillin-resistant control strain | 8 | 4 |
| Staphylococcus aureus - methicillin-resistant clinical isolate | 8 | 4 |
| Staphylococcus aureus - multi-drug-resistant clinical isolate | 16 | 4 |
| Staphylococcus aureus - teicoplanin-intermediate clinical isolate | 16 | 4 |
| Staphylococcus epidermidis antibiotic susceptible clinical isolate | 4 | 8 |
| Staphylococcus epidermidis methicillin-resistant clinical isolate | 4 | 2 |
| Staphylococcus haemolyticus - antibiotic susceptible clinical isolate | 4 | 4 |
| Staphylococcus saprophyticus - antibiotic susceptible clinical isolate | 2 | 0.5 |
| Enterococcus faecalis - ATCC 29212 antibiotic-susceptible control strain | 16 | 4 |
| Enterococcus faecalis vancomycin-susceptible clinical isolate | 32 | 4 |
| Enterococcus faecalis vancomycin-resistant (VanA) clinical isolate | 32 | 4 |
| Enterococcus faecalis vancomycin-resistant (VanB) clinical isolate | ≥128 | 8 |
| Enterococcus faecalis high-level gentamicin-resistant clinical isolate | 64 | 8 |
| Enterococcus faecium vancomycin-susceptible clinical isolate | 16 | 4 |
| Enterococcus faecium vancomycin-resistant (VanA) clinical isolate | 32 | 4 |
| Enterococcus faecium vancomycin-resistant (VanB) clinical isolate | 16 | 4 |
| Enterococcus gallinarum vancomycin-resistant (VanC) clinical isolate | 8 | 4 |
| Streptococcus pneumoniae - ATCC 49619 antibiotic-susceptible control strain | 32 | 16 |
| Streptococcus pneumoniae - penicillin-susceptible clinical isolate | 32 | 8 |
| Streptococcus pneumoniae - penicillin-intermediate clinical isolate | 32 | 16 |
| Streptococcus pneumoniae - penicillin-resistant clinical isolate | 32 | 16 |
| Streptococcus pneumoniae - multi-drug resistant clinical isolate | 32 | 16 |
| Streptococcus pyogenes - Macrolide (MLS) resistant clinical isolate | 16 | 8 |
| Streptococcus pyogenes - Macrolide (M-type) resistance clinical isolate | 16 | 8 |
| Corynebacterium jeikeium - antibiotic-susceptible clinical isolate | 8 | 4 |
| Corynebacterium jeikeium - multi-drug resistant clinical isolate | 8 | 2 |
| Listeria monocytogenes - antibiotic-susceptible clinical isolate | 16 | 8 |
| MU50 Staphylococcus aureus (MRSA) - VISA type strain | 16 | 4 |
| EMRSA3 Staphylococcus aureus (MRSA) - SSCmec type 1 | 8 | 4 |
| EMRSA16 Staphylococcus aureus (MRSA) - SSCmec type 2 | 16 | 4 |
| EMRSA1 Staphylococcus aureus (MRSA) - SSCmec type 3 | 16 | 4 |
| EMRSA15 Staphylococcus aureus (MRSA) - SSCmec type 4 | 8 | 4 |
| HT2001254 Staphylococcus aureus (MRSA) - PVL positive | 8 | 4 |
| Streptococcus agalactiae - antibiotic-susceptible clinical isolate | 8 | 8 |
| Streptococcus agalactiae - macrolide-resistant clinical isolate | 16 | 8 |
| Group C Streptococcus - antibiotic-susceptible clinical isolate | 16 | 8 |
| Group C Streptococcus - macrolide-resistant clinical isolate | 32 | 16 |
| Group G Streptococcus - antibiotic-susceptible clinical isolate | 16 | 8 |
| Group G Streptococcus - macrolide-resistant clinical isolate | 16 | 8 |
| Streptococcus mitis - antibiotic-susceptible clinical isolate | 32 | 16 |
| Streptococcus mitis - macrolide-resistant clinical isolate | 64 | 16 |
| Streptococcus constellatus - antibiotic-susceptible clinical isolate | 64 | 16 |
| Streptococcus constellatus - macrolide-resistant clinical isolate | 32 | 16 |
| Streptococcus oralis - antibiotic-susceptible clinical isolate | 64 | 16 |
| Streptococcus oralis - macrolide-resistant clinical isolate | 64 | 16 |
| Streptococcus bovis - antibiotic-susceptible clinical isolate | 32 | 8 |
| Streptococcus bovis - macrolide-resistant clinical isolate | 8 | 2 |
| Streptococcus sanguis - antibiotic-susceptible clinical isolate | 32 | 16 |
| Streptococcus sanguis - macrolide-resistant clinical isolate | 32 | 16 |
| Clostridium perfringens - antibiotic-susceptible clinical isolate | ≥128 | 32 |
| Clostridium difficile - antibiotic-susceptible clinical isolate | 64 | 32 |
| Propionibacterium acnes- antibiotic-susceptible clinical isolate | | 4 |
| Propionibacterium acnes- antibiotic-resistant clinical isolate | | 2 |

### EXAMPLE 7

### In Vivo Activity of Compounds 7 and 8

The skin of mice was infected with Staphylococcus aureus or Streptococcus pyogenes and subsequently given a total of three treatments at three hourly intervals. Three hours after the last treatment, skin biopsies were collected and the number of colony forming units (CFUs) present in the skin sample was determined. Results are shown in FIGS. 3, 4 and 5 expressed as the number of colony forming units per mouse.

In experiment 1 (FIG. 3), compound 7 was applied to the murine skin as part of either a cream or a gel containing 2% (w/w) of compound 7. The same cream or gel without compound 7 was used as a negative control (placebo). Bactroban 2% cream was used as a positive control. It can clearly be seen that the number of CFUs was reduced when a cream or gel containing compound 7 was applied to the murine skin, compared to the negative control, indicating that compound 7 exerted an antimicrobial effect against Staphylococcus aureus. The efficacy of standard clinical treatment, Bactroban 2% cream, had no significant effect under the treatment regime. The nature of the carrier, cream or gel, had no significant effect.

In experiment 2 (FIG. 4), compound 7 was applied in two different concentrations, as either a 1% or a 2% gel. A placebo gel and a known antibacterial “bactroban (mupericin)” were used as controls. It can be seen that gels containing compound 7 were more effective at reducing the number of CFUs from a Streptococcus pyogenes CS 301 infection than the placebo gel or the bactroban. The gel containing 2% of compound 7 was more effective than the gel containing only 1% of compound 7.

In experiment 3 (FIG. 5), compound 8 was applied in a 2% cream formulation on a Staphylococcus aureus FDA 486 infection in the murine skin infection model. A placebo cream and two known antibacterials, “Fucidin (fucidic acid) ointment 2%” and “Bactroban (mupericin) cream 2%” were used as controls. It can be seen that a cream containing compound 8 was more effective at reducing the number of CFUs than the placebo and fucidin or bactroban.

### EXAMPLE 8

### Preparation of Absorbable Braided Sutures with AMC-109 Containing Coating

The purpose was to investigate solution coating of braided sutures with a typical biodegradable poly(lactide-co-glycolide) polymer containing AMC-109. The work was in four areas:
1. Preparing a solvent (or solvent mixture) allowing dissolution of the poly(lactide-co-glycolide) polymer and AMC-109
2. Coat “naked” braided absorbable suture material with the solution from item 1. above
3. Investigate the leakage of AMC-109 from the coated suture
4. Investigate the microbiological efficacy of the coated suture

### Material and Methods

Polymer

Resomer® RG 502, Poly(D,L-Lactide-co-Glycolide) Sigma-Aldrich no. 719889 is a lactide:glycolide 50:50 mixture, ester terminated, Mw 7,000-17,000, biodegradable polymer. It is similar to biodegradable polymers generally used to coat commercial absorbable sutures. Its chemical structure is shown below:

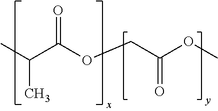

Peptide

AMC-109 is referred to herein as “Compound 2” and has the formula Arg-Tbt-Arg-NHCH₂CH₂Ph.

Suture

Syneture Surgilon size 4-0, a nylon braided suture with silicon coating was used in the investigations.

Procedure for the Preparation of Coated Sutures

The suture was washed with ethyl acetate to remove most of the pre-existing coating layer before coating.

Resomer® RG 502 was dissolved in ethyl acetate and AMC-109 was dissolved in ethanol. In the first test, Test-01, 50 mg of RG 502 was dissolved in 300 μl ethyl acetate and mixed with 10 mg AMC-109 dissolved in 50 μl ethanol. In the second test, Test-02, 50 mg of RG 502 was dissolved in 400 μl ethyl acetate and mixed with 10 mg AMC-109 dissolved in 110 μl ethanol. The resulting solvent mixtures were homogeneous.

The suture base material (after ethyl acetate washing) was coated by dipping repeatedly into the coating solvent mixture.

Microbiology
Bacterial strain: Staphylococcus aureus
Sutures used for (colony forming unit) CFU counts:
   Suture Surgilon 4-0 with RG502 polymer, control
   Suture Surgilon 4-0 with RG502 polymer/AMC-109
   Suture Surgilon 4-0 (naive), control
Liquid Tryptic Soy Broth (TSB)
Mueller Hinton (MH) agar plates

Colonies of S. aureus were picked from a blood agar plate, and a 0.5 McFarland solution was made (1×10⁸ CFU), this solution was used to:
1. Inoculate MH agar plates
2. Make dilutions in TSB to obtain 1×10⁵ CFU

Sutures were placed on the agar plates to observe the zone of inhibition and inoculated in TSB to examine antibacterial effect of the coated suture vs. the uncoated sutures.

Samples were incubated for 16 hours at 37° C.

The cultures from tubes with sutures inoculated in TSB were used for CFU determination by serial dilutions. To investigate the long-term effect of the coated sutures, sutures were rinsed and re-inoculated with bacteria in TSB for 16 hours at 37° C.

Three parallel experiments were performed.

CFU was determined after colony counts.

### Results

Preparation of Coated Sutures

Coated sutures could readily be produced by treating the sutures with a solution of Resomer RG-502 and AMC-109. The composition of the solvent mixture was critical to the ability to dissolve both Resomer RG-502 and AMC-109 and mix (and avoid phase separation or precipitation).

Quantitative Analysis of the Amount of AMC-109 Released

The coated sutures were extracted with water. The coated suture was placed in 0.4 ml water and left standing for 30 min. The sutures were removed, dried and extracted a second time, for 22 h. The extracts were analysed for the amount AMC-109 that was released by the extraction. The results are shown in FIG. 6.

### Determination of the Antimicrobial Efficacy of AMC-109 Coated Sutures

The antimicrobial efficacy of the sutures coated by Resomer RG-502 and AMC-109 was assessed by an agar growth inhibition assay and a liquid broth assay.

Day 1:

A zone of inhibition was observed around the sutures coated with AMC-109, compared to a naive control suture and a suture coated with RG-502 (no AMC-109 added) (FIG. 7).

At day one there was no obvious growth in the TSB media in the tubes containing AMC-109 coated sutures. Dilutions for CFU counts were made, obtaining a CFU count of 0, 500 and 3×10³ for the AMC-109 coated sutures. In the tubes containing the controls there was visible growth, resulting in CFU counts of 5.8×10⁸ and 4.7×10⁸ (numbers are averages of the parallels) (FIG. 8).

Day 2:

At day two there was visible growth in all tubes, and there was no visible difference between tubes with Surgilon RG502/AMC and the controls.

### Conclusion

Sutures can readily be coated by a solution of a biodegradable polymer and AMC-109. The resulting sutures coated with AMC have an antibacterial effect over a period of 16 hours. A final Resomer layer without AMC-109 could be added to reduce the immediate diffusion, providing a more lasting effect.

### EXAMPLE 9

### Sutures with AMC-109/Polymer Coating

### Materials and Methods

Bacterial strains: S. aureus ATCC29213 and S. epidermidis RP42A

Sutures: Ethibond® Excel polymer suture (Johnson & Johnson) was covered with a coating of polycaprolactone+5% AMC-109. The polymer (i.e. polycaprolactone, average molecular weight (MVV) approx. 14,000) and peptide were melted by heating rapidly (over about 3 minutes) to 120° C. in a glass vial and mixing was done when the polycaprolactone was melted. The suture was dipped in the molten mixture to coat it.

Controls: uncoated Ethibond® Excel sutures

In one experiment bacterial colonies were diluted to 0.5 McFarland and spread on Mueller Hinton agar plates and in a second experiment the colonies were diluted to 0.5 McFarland and diluted 1:100 in Tryptic Soy Broth (TSB).

In the first experiment AMC coated sutures and uncoated controls were placed on inoculated plates. Plates were incubated at 37° C. for 16 hours. In the second experiment AMC coated sutures and uncoated controls were placed in inoculated media and incubated with shaking at 37° C. for 16 hours.

Results

A clear zone of inhibition was observed on the agar plates around the AMC-109 coated suture compared to control for both S. epidermidis and S. aureus (FIG. 9).

There was a clear inhibition of bacterial growth observed in wells 1 and 4 (FIG. 10) to which had been added a portion of an Ethibond suture coated with AMC.

Sutures were further stained with Syto 9 and Propidium iodide and investigated by fluorescence microscopy. There was a clear distinction between the coated and uncoated sutures; massive bacterial growth was observed on the uncoated sutures.

### EXAMPLE 10

### Absorbable Sutures Coated with AMC-109

The process described is scalable and suitable for industrial development.

### Materials and Methods

Sutures
Coviden (Medtronic) Polysorb 3-0
Ethicon Vicryl plus 3-0 (triclosan containing, positive control)

Both sutures consist of an inner braided filament of Polyglactin 910, that is covered in an outer, softer and lubricating layer consisting of a poly(D,L-lactide-co-glycolide) (lactide:glycolide 65:35) mixed with calcium stearate. The Vicryl plus suture contains additionally triclosan as an active ingredient in the outer layer.

Polymer

Rsomer (Evonik) RG-502, degradable poly(D,L-lactide-co-glycolide) (lactide:glycolide 50:50) M_{w} 7000-17000, degradation time<3 months.

Peptide

AMC-109 as before.

Suture Stripping

The outer layer of the Polysorb sutures was (partially) removed be washing the suture with ethyl acetate for 10 min followed by water for 2 min. The sutures were dried before coating.

Suture Coating Mixture

The coating mixture was prepared by dissolving RG-502 in ethyl acetate in one vial and dissolving AMC-109 in ethanol in a second vial. The two solutions were mixed generating a slightly turbid solution that became clear upon adding 0.5 ml ethyl acetate.

Suture Coating

The Polysorb sutures (cut in pieces of 6 cm) were soaked for 10 min in the coating mixture and dried before a second soaking for 2 min in a freshly prepared coating mixture.

Two batches of sutures were prepared, one for chemical extraction analysis and zone of inhibition testing (experiment 1) and a second for efficacy test in liquid media (experiment 2).

TABLE 7
Masses and volumes used in the coatings.

| | RG502 | EtOAc | AMC-109 | EtOH | | |
|---|---|---|---|---|---|---|
| Suture | (mg) | (ml) | (mg) | (ml) | EtOAc | Turbidity |
| Exp. 1 15% | 45.00 | 0.5 | 8 | 0.25 | 0.5 | Turbid |
| Exp. 1 5.9% | 77.68 | 1.907 | 4.90 | 0.25 | 0 | “Clear” |
| Exp. 1 | 50.00 | 1.00 | 0 | 0.25 | 0 | |
| control | | | | | | |
| Exp. 2 15% | 90.00 | 1.5 | 16.1 | 0.5 | 0.5 | Turbid |
| Exp. 2 6.1% | 90.00 | 1.5 | 5.9 | 0.5 | 0.5 | Turbid |

Extraction

A sample of sutures were selected for aqueous extraction.

First Extraction

The suture was placed in a vial and water (1 ml) was added. The samples were left for 1 h. The extraction samples were analysed by HPLC.

Second Extraction

The suture sample from the first extraction was placed in a new vial and water (1 ml) was added and extracted for 3.5 h. The second extraction samples were analysed by HPLC.

Microbiological Evaluation

Bacterial Strains:
Staphylococcus aureus (8325)
Pseudomonas aeruginosa (PAO1)
Escherichia coli
Enterococcus faecium

Overnight colonies of the different bacterial strains were used to make 0.5 McFarland (1×10⁸ CFU/ml) solutions in 0.5% NaCl and further diluted in tubes with 2 ml LB (Lysogeny broth) to 10⁵ CFU/ml. The bacterial solution was used for inoculation of agar plates for the growth zone inhibition test, and for direct inoculation of suture test samples in LB media. All samples were incubated at 37° C. for 18 hours. Enumeration of CFU was performed by making serial dilutions from i) solution in which the test samples were inoculated (LB-media), or from ii) solution (1 ml NaCl, 0.5%) with rinsed and vortexed (20 s) samples. Serial dilutions (10⁻¹-10⁻⁶) were made in 1 ml NaCl. From the different dilutions, 100 μl was spread on blood agar plates and further incubated at 37° C. for 18 hours.

Results:

Quantification of AMC-109 Release

TABLE 8
Amount (μg/ml) of AMC-109 found in the aqueous suture extracts.

| AMC-109 | First extraction | Second extraction |
|---|---|---|
| 15% | 21 | 4 |
| 5.9% | 5 | NQ |

*NQ denotes not quantifiable

The sutures gained between 0.4 and 0.5 mg upon coating. The AMC-109 concentration in the aqueous extracts are compiled in Table 8, and the data revealed that the high loading suture liberates 25 microgram AMC-109 into the aqueous solution upon extraction for a total of 4.5 h. The low loading suture released 5 microgram AMC-109 under similar conditions.

The amounts of AMC-109 suggest that at least 30% of the AMC-109 embedded in the high loading coating is released into water within 4.5 h. The release from the low loading suture seems to be slightly lower.

Microbiological Assessment

A zone of growth inhibition was always observed around Polysorb coated with 15% AMC-RG502. In comparison, Vicryl Triclosan only showed growth inhibition of S. aureus and E. coli, Table 9.

TABLE 9
Zone inhibition test

| | | Inhibition zone | | | |
|---|---|---|---|---|---|
| | | S. aureus | P. aeruginosa | E. faecium | E. coli |
| Exp. 1 | Polysorb | 4 mm | 2 mm | 3 mm | 3 mm |
| 15% | 3-0 | | | | |
| Exp. 1 | Polysorb | 2 mm | 0 mm | 1 mm | 0 mm |
| 5.9% | 3-0 | | | | |
| Exp. 1 | Polysorb | 0 mm | 0 mm | 0 mm | 0 mm |
| control | 3-0 | | | | |
| Triclosan | Vicryl | 15 mm | 0 mm | 0 mm | 3 mm |

Direct inoculation of the suture test material in bacterial suspension resulted in bacterial growth reduction in LB media for sutures coated with 15% AMC-109 for all strains. Triclosan coated sutures inhibited growth of S. aureus only, Table 10.

TABLE 10
CFU from direct inoculation of suture test samples in LB media.

| | | Colony forming units (CFU) | | | |
|---|---|---|---|---|---|
| | | S. aureus | P. aeruginosa | E. faecium | E. coli |
| Exp. 2 | Polysorb | 0 | 3.4 × 10⁵ | 0 | 0 |
| 15% | 3-0 | | | | |
| Exp. 2 | Polysorb | 3.2 × 10⁶ | 1.0 × 10⁹ | 1.6 × 10³ | 6 × 10⁸ |
| 6.1% | 3-0 | | | | |
| Control | Polysorb | 6.0 × 10⁸ | 1.0 × 10⁹ | 4.6 × 10⁷ | 1.5 × 10⁸ |
| | 3-0 | | | | |
| Triclosan | Vicryl | 80 | 1.2 × 10⁹ | 1.5 × 10⁷ | 8.0 × 10⁷ |

### Conclusion

AMC-109 can be incorporated into absorbable sutures using the described solution coating technique. The coating solution is turbid suggesting a supersaturated solution. This may increase coating efficiency.

The AMC-coated sutures liberated at least 30% of their AMC-109 content into an aqueous environment within 4.5 h. The released amount of AMC-109 is dependent on the amount compounded into the suture.

AMC-109 coated sutures provide anti-colonizing efficacy against a range of Gram-positive and Gram-negative bacteria, including important pathogens where triclosan is ineffective.

### EXAMPLE 11

### Casting of Bioresorbable Thin Films Containing AMC-109

### 11.1 Bioresorbable Polymers

Resomer L206S (poly L-lactide ester terminated) (Sigma Aldrich 719854) dissolved in dichloromethane (DCM), AMC-109 dissolved in chloroform
Resomer RG502 (poly-D,L-lactide-co-glycolide) (Sigma Aldrich 719889) dissolved in tetrahydrofuran (THF), AMC-109 also dissolved in THF

### 11.2 Preparation of Bioresorbable Thin Films

Casting Solution

The Resomer RG502 bioresorbable polymer material and AMC-109 were separately dissolved in THF in such a manner that amount of AMC-109 compared to the bioresorbable polymer in the final painting solution was 5%. The dissolution of AMC-109 in THF takes several hours.

The Resomer L2065 bioresorbable polymer material and AMC-109 were separately dissolved in dichloromethane and chloroform, respectively. The ratio between AMC-109 and L2065 was the same as above.

Casting Process

The thin film samples were prepared by placing 8 ml of the casting solution on aluminium foil with a shallow indentation or pouring the painting solution on a watch glass. After drying (several days), the thin film casting film was mechanically loosened from its surface.

### 11.3 Determination of AMC-109 Leakage from Bioresorbable Thin Films Containing AMC-109

Extraction

A sample was cut from the cast thin film and accurately weighed (100-150 mg), and the amount of AMC-109 in the sample was calculated. The samples were placed in vials, water (2 ml) was added and the vials shaken. Six consecutive extractions were performed. For each extraction the old extract was replaced by deionized water (2 ml). The extractions were performed with a shaking period of 10 s, 5 min, 30 min, 3 h, 22 h, and 48 h. The amount of AMC-109 in each extract was determined by UV-spectrophotometry at 280 nm using a pre-made standard curve. The results are shown graphically in FIG. 11.

### 11.4 Microbiological Evaluation of Bioresorbable Thin Films Containing AMC-109

Bacterial Strains:
Staphylococcus aureus 8325

Modified AATCC-100 Method

Overnight colonies of S. aureus were diluted to 0.5 McFarland in 0.9% NaCl resulting in a bacterial concentration of 1×10⁸ bacteria. This solution was further diluted in TSB to 1×10⁵ bacteria.

The thin film material of L2065 was cut in pieces of approximately 0.4×0.4 cm. The material was then submerged in dH₂O for 2 minutes and air dried before use. The samples were inoculated with 50 μl of the bacterial solution (1'10⁵). The samples were placed on a glass slide and incubated in a moisture chamber at 37° C. for 24 hours. Two biological replicates of each test material were made.

After incubation the thin film material was washed thoroughly for 2 minutes to remove AMC-109 that is readily extractable or residing directly on the surface. Then it was placed in 1000 μl NaCl and vortexed for 45 seconds, before making serial dilutions (0-10⁻⁶) and plating of 100 μl for CFU counting.

Microbiological Efficacy

Colony Forming Units

The number of CFU were below the detection limit for the AMC containing material. Compared to the control material there was a 7 log reduction in CFU numbers, see Table below.

TABLE
CFU values for bioresorbable thin
film samples containing AMC-109.

| TPU/polymer | AMC-109 | Control |
|---|---|---|
| L206S | 0 | 8.7 × 10⁷ |
| RG502 | * | * |

*the RG502 material disintegrated and adhered strongly to the glass slide surface during the 24 h incubation period. The material could not be recovered.

### 11.5 Conclusion

Resomers can be dissolved by some solvents, THF and dichloromethane has the most general applicability among the solvents tested.
AMC-109 can be mixed into the dissolved polymers either in THF or chloroform solution. The solvent dissolving the AMC-109 must be miscible with the solvent used to dissolve the Resomers.
The resulting casting solution can be applied on several surfaces or be used for casting thin films.
The casted thin films leak AMC-109, rapidly at first, sustained at a lower concentration over at least two days depending on the nature of the Resomer.
The Resomer RG502 thin film shows an interesting leakage behaviour. The polymer disintegrates over time and continuously leaks AMC-109 over a prolonged period.
The Resomer L206S thin film does not disintegrate as rapidly as Resomer RG502 and may have activity for a longer period of time.

1. A formulation comprising a biodegradable polyester compounded with a compound of Formula (I):
AA-AA-AA-X-Y (I)
wherein, in any order, 2 of said AA (amino acid) moieties are cationic amino acids and 1 of said AA is an amino acid with a lipophilic R group, the R group having 14-27 non-hydrogen atoms;
X is a N atom, which may be substituted by a branched or unbranched C₁-C₁₀ alkyl or aryl group, which group may incorporate up to 2 heteroatoms selected from N, O and S; and
Y is selected from the group consisting of R₁-R₂-R₃,
R₁-R₂-R₂-R_{3,} R₂-R₂-R₁-R₃, R₁-R₃ and R₄
wherein:
R₁ is C, O, S or N,
R₂ is C;
each of R₁ and R₂ may be substituted by C₁-C₄ alkyl groups or unsubstituted;
R₃ is a group comprising 1 to 3 cyclic groups each of 5 or 6 non-hydrogen atoms, 2 or more of the cyclic groups may be fused and one or more of the cyclic groups may be substituted; R₃ incorporates a maximum of 15 non-hydrogen atoms; and
R₄ is an aliphatic moiety having 2-20 non-hydrogen atoms, said moiety being linear branched or cyclic.

2. The formulation of claim 1, wherein said compound is a peptide.

3. The formulation of claim 1, wherein said cationic amino acids are arginine and/or lysine.

4. The formulation of claim 1, wherein said lipophilic R group comprises 2 or more cyclic groups which may be fused or connected.

5. The formulation of claim 1, wherein said amino acid with a lipophilic R group is selected from tributyl tryptophan (Tbt) or a biphenylalanine derivative selected from Phe (4-(2-Naphthyl)), Phe (4-(1-Naphthyl)), Bip (4-n-Bu), Bip (4-Ph) or Bip (4-T-Bu).

6. The formulation of claim 1, wherein said compound is a compound of formula (II)
AA₁-AA₂-AA₁-X-Y (II)
wherein:
AA₁ is a cationic amino acid;
AA₂ is an amino acid with a lipophilic R group, the R group having 14-27 non-hydrogen atoms; and
X and Y are as defined in claim 1.

7. The formulation of claim 1, wherein said compound has the structural formula

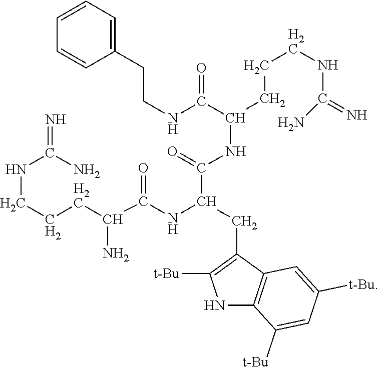 [SMILES: CC(C)(C)c1cc(C(C)(C)C)c2[nH]c(C(C)(C)C)c(CC(NC(=O)C(N)CCCNC(=N)N)C(=O)NC(CCCNC(=N)N)C(=O)NCCc3ccccc3)c2c1]

8. The formulation of claim 1, wherein said polyester is selected from the group consisting of a polylactide, polyglycolide, polydioxanone and polycaprolactone and co-polymers thereof.

9. The formulation of claim 1, wherein said polyester has an intrinsic viscosity between 0.1 and 8 dL/g.

10. The formulation of claim 1, wherein said polyester has a molecular weight between 3,000 and 30,000.

11. The formulation of claim 1, wherein said polyester is poly(D,L-lactide-co-glycolide), poly(L-lactide) or polycaprolactone.

12. The formulation of claim 1 which is a controlled release formulation.

13. The formulation of claim 1 wherein the compound of Formula (I) is releasably dispersed through the polyester.

14. A medical device comprising or consisting of a formulation of claim 1, preferably a medical device comprising said formulation as a coating thereon.

15. The device of claim 14, wherein said medical device is a surgical fastener or implant.

16. The device of claim 15, wherein said medical device is a suture.

17. A method of producing a formulation as defined in claim 1, said method comprising melting the biodegradable polyester in admixture with a compound of Formula (I).

18. A method of producing a formulation as defined in claim 1, said method comprising forming a mixture of a compound of Formula (I), a biodegradable polyester and one or more solvents capable of dissolving said compound and said polyester.

19. The method of claim 18, said method comprising (i) providing a first solution comprising a compound of Formula (I); (ii) providing a second solution comprising a biodegradable polyester, wherein said second solution is miscible with the first solution; and (iii) mixing said first and second solutions.

20. A method of producing a medical device as defined in claim 14, said method comprising (i) providing a formulation; and (ii) applying said formulation to a medical device.

21. The method of claim 20, wherein the formulation is applied by dipping the device into the formulation or painting the formulation onto the device.

22. The method of claim 20, wherein after application of the formulation to the medical device it is dried.

23. (canceled)

24. (canceled)

25. A method of treating or preventing an infection which method comprises contacting a subject in need thereof with a therapeutically effective amount of a formulation as defined in claim 1.
